SEQUENCE LISTING (49 sequences)

SEQ ID NO: 1 - protein, 58 aa, Human cytomegalovirus
STSHATSSTHNGSHTSRTTSAQTRSVYSQHVTSSEAVSHRANETIYNTTLKYGDVVGV

SEQ ID NO: 2 - protein, 58 aa, Human cytomegalovirus
STRGTSATHSHHSSHTTSAAHSRSGSVSQRVTSSQTVSHGVNETIYNTTLKYGDVVGV

SEQ ID NO: 3 - protein, 129 aa, Human cytomegalovirus
LLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLK

SEQ ID NO: 4 - DNA, 396 bp, Homo sapiens
gaggtgcagctggtgcaatctggagcagaggtgaaaaagcccggggagtctctgaagatctcctgtaagggttctggatacacctttgccaactactggatcgcctgggtgcgccagatgcccgggaaaggcctggagtggatgggaatcatctatcctggtgactctgataccagatacagcccgtccttccaaggccaggtcaccatctcagccgacaagtccttccacaccgtctacctgcagtggagcagcctgaaggcctcggacaccgccatctatttctgtgcgagcacctcgtattacgacttttggagtggttatcatagggggcgagaacactactactacggtatggaagtctggggccaagggaccacggtaatcgtctcctca

SEQ ID NO: 5 - protein, 132 aa, Homo sapiens
EVQLVQSGAEVKKPGESLKISCKGSGYTFANYWIAWVRQMPGKGLEWMGIIYPGDSDTRYSPSFQGQVTISADKSFHTVYLQWSSLKASDTAIYFCASTSYYDFWSGYHRGREHYYYGMEVWGQGTTVIVSS

SEQ ID NO: 6 - protein, 8 aa, Homo sapiens
GYTFANYW

SEQ ID NO: 7 - protein, 7 aa, Homo sapiens
IYPGDSD

SEQ ID NO: 8 - protein, 25 aa, Homo sapiens
ASTSYYDFWSGYHRGREHYYYGMEV

SEQ ID NO: 9 - DNA, 312 bp, Homo sapiens
acacagtctccagccaccctgtctttgtctccaggagaaagagtcaccctctcctgcagggccagtcagagtgtttacaactacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcggtttattactgtcagctgcgtcgagggacgttcggccaagggaccaaggtggagatcaaacgaactgtggct

SEQ ID NO: 10 - protein, 104 aa, Homo sapiens
TQSPATLSLSPGERVTLSCRASQSVYNYLAWYQQKPGQAPRLLIYDASNRATGIPARFSGSGSGTDFTLTISSLEPEDFAVYYCQLRRGTFGQGTKVEIKRTVA

SEQ ID NO: 11 - protein, 6 aa, Homo sapiens
QSVYNY

SEQ ID NO: 12 - protein, 3 aa, Homo sapiens
DAS

SEQ ID NO: 13 - protein, 5 aa, Homo sapiens
QLRRG

SEQ ID NO: 14 - DNA, 357 bp, Homo sapiens
caggttcagttggtacagtctggaggtgaggtgaagaagcctggggcctcagtgaaggtctcctgcaaggcttctggttacacctttaccagctctggtatcaactgggtgcgacaggcccctggacaaggacttgagtggatgggatggatcaacacttacaatggtaacacaaagtatgcacagaggctccagggcagagtcaccatgaccacagacacatccacggacacagcctacatggagctgaggagcctgagatctgacgacacggccgtgtattactgtgcgagagatggcctaaactgggggtggggggactactggggccagggaaccctggtcaccgtctcctca

SEQ ID NO: 15 - protein, 119 aa, Homo sapiens
QVQLVQSGGEVKKPGASVKVSCKASGYTFTSSGINWVRQAPGQGLEWMGWINTYNGNTKYAQRLQGRVTMTTDTSTDTAYMELRSLRSDDTAVYYCARDGLNWGWGDYWGQGTLVTVSS

SEQ ID NO: 16 - protein, 8 aa, Homo sapiens
GYTFTSSG

SEQ ID NO: 17 - protein, 7 aa, Homo sapiens
INTYNGN

SEQ ID NO: 18 - protein, 12 aa, Homo sapiens
ARDGLNWGWGDY

SEQ ID NO: 19 - DNA, 321 bp, Homo sapiens
acccagtctccttccaccctgtctgcatctgtaggagacagagtcattatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaagttattactgccaacagtgtaaaggttacccgtacacttttggccagggcaccaagctggaaatcaaacggactgtggct

SEQ ID NO: 20 - protein, 107 aa, Homo sapiens
TQSPSTLSASVGDRVIITCRASQSISSWLAWYQQKPGKAPKLLIYKASSLESGVPSRFSGSGSGTEFTLTISSLQPDDFASYYCQQCKGYPYTFGQGTKLEIKRTVA

SEQ ID NO: 21 - protein, 6 aa, Homo sapiens
QSISSW

SEQ ID NO: 22 - protein, 3 aa, Homo sapiens
KAS

SEQ ID NO: 23 - protein, 8 aa, Homo sapiens
QQCKGYPY

SEQ ID NO: 24 - protein, 131 aa, Homo sapiens
QVQLQESGPGLVKPSQTLSLICTVSGGSVSSGGDYWTWIRQHPGKGLEWLGYIHSSGNIFYNPSLKSRLTLSMDTSKNQFFLKLTSVTAADTAVYYCARVYHKDFVVVPGAFPFEFWFDPWGQGTLVTVSS

SEQ ID NO: 25 - protein, 109 aa, Homo sapiens
TQPPSVSVAPGQTARIPCGGNEIGSKSVHWYQQKPGQAPVLVVHDDSDRPSGIPDRFSGSNSGNTATLTISRVEAGDEADYYCQVWDSGSDHHVVFGGGTKLTVLGQPK

SEQ ID NO: 26 - DNA, 372 bp, Homo sapiens
gaggtgcagctggtggagtctgggggaggcttggtacagccagggcggtccctgagactctcctgtacagcttctagattcacgtttgctgattatgctatcacctgggtccgccaggctccagggaaggggctggagtgggtaggtttcattagaagcaaaccttttggtgggacagcagcatacgccgcgtctgtgaaaggcagattcaccatctcaagagatgattccaaaagcatcgcctatctgcaattgaaccgcctgaaaaccgaagacacagccgtgtattactgtagtagagcccctttatcgagtgactacagtccctccttggacgtctggggccaagggaccacggtcaccgtctcctca

SEQ ID NO: 27 - protein, 124 aa, Homo sapiens
EVQLVESGGGLVQPGRSLRLSCTASRFTFADYAITWVRQAPGKGLEWVGFIRSKPFGGTAAYAASVKGRFTISRDDSKSIAYLQLNRLKTEDTAVYYCSRAPLSSDYSPSLDVWGQGTTVTVSS

SEQ ID NO: 28 - protein, 8 aa, Homo sapiens
RFTFADYA

SEQ ID NO: 29 - protein, 9 aa, Homo sapiens
IRSKPFGGT

SEQ ID NO: 30 - protein, 15 aa, Homo sapiens
SRAPLSSDYSPSLDV

SEQ ID NO: 31 - DNA, 336 bp, Homo sapiens
actcagtctccactctccctgcccgtcacccctggagagccggcctccatctcctgcaggtctagtcagagcctcctgcatagtaatggatacaactatttggattggtacgtgcagaagccagggcagtctccacagctcctgatcaatttgggttctaatcgggcctccggggtccctgacaggttcagtggcagtggatcaggcacagattttacactgaaaatcagcagagtggaggctgaggatgttggggtttattactgcatgcaaggtctacaaactcctcacacttttggccagggcaccaagctggaaatcaaacggactgtggct

SEQ ID NO: 32 - protein, 112 aa, Homo sapiens
TQSPLSLPVTPGEPASISCRSSQSLLHSNGYNYLDWYVQKPGQSPQLLINLGSNRASGVPDRFSGSGSGTDFTLKISRVEAEDVGVYYCMQGLQTPHTFGQGTKLEIKRTVA

SEQ ID NO: 33 - protein, 11 aa, Homo sapiens
QSLLHSNGYNY

SEQ ID NO: 34 - protein, 3 aa, Homo sapiens
LGS

SEQ ID NO: 35 - protein, 8 aa, Homo sapiens
MQGLQTPH

SEQ ID NO: 36 - DNA, 384 bp, Homo sapiens
caggtgcagctggtggagtctgggggaggcgtggtccagcctgggaagtccctgagactctcctgtgcagcctctggattcaccttcagtagctatggcatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtataactgtgcgaaagagatgaggtatggttcggggagaaaagcgattaaatactactacggtatggacgtctggggccaagggaccacggtcaccgtctcctca

SEQ ID NO: 37 - protein, 128 aa, Homo sapiens
QVQLVESGGGVVQPGKSLRLSCAASGFTFSSYGMYWVRQAPGKGLEWVAVISYDGSNKYYADSVKGRFTISRDNSKNTLYLQMNSLRAEDTAVYNCAKEMRYGSGRKAIKYYYGMDVWGQGTTVTVSS

SEQ ID NO: 38 - protein, 8 aa, Homo sapiens
GFTFSSYG

SEQ ID NO: 39 - protein, 7 aa, Homo sapiens
ISYDGSN

SEQ ID NO: 40 - protein, 21 aa, Homo sapiens
AKEMRYGSGRKAIKYYYGMDV

SEQ ID NO: 41 - DNA, 336 bp, Homo sapiens
acccaacctctactctccctgcctgtcagtcttggagatcaagcctccatctcttgcagatctagtcagagcattgtacatagtaatggaaacacctatttagaatggtacctacagaaaccaggccagtctccaaagctcctgatccacagagtttcctaccgattttctggggtcccagacaggttcagtggcagtggatcagggacagatttcacactcgtgatcagcagagtggaggctgaggatctgggagtttattactgctttcagggttcacatgttccattcacgttcggctcgggcaccaagctggaaatcaaacggactgtggct

SEQ ID NO: 42 - protein, 112 aa, Homo sapiens
TQPLLSLPVSLGDQASISCRSSQSIVHSNGNTYLEWYLQKPGQSPKLLIHRVSYRFSGVPDRFSGSGSGTDFTLVISRVEAEDLGVYYCFQGSHVPFTFGSGTKLEIKRTVA

SEQ ID NO: 43 - protein, 11 aa, Homo sapiens
QSIVHSNGNTY

SEQ ID NO: 44 - protein, 3 aa, Homo sapiens
RVS

SEQ ID NO: 45 - protein, 8 aa, Homo sapiens
FQGSHVPF

SEQ ID NO: 46 - protein, 126 aa, Homo sapiens
QVQLQESGGGVVQPGRSLRLSCAASGFTFSTYGMHWVRQAPGKGLEWVAVISYDGSSKYSADSVKGRFTISRDNFKNTVYLQMNSLRAEDTAVYYCAKDNYSKYGVVRVGYGMDVWGQGTTVTVSS

SEQ ID NO: 47 - protein, 124 aa, Homo sapiens
EVQLVQSGAEVKKPGESLKISCKGSGYTFDSYWIGWVRQMPGKGLEWMGIIYPGDSDTRYSPSFQGQVTISADKSISTASLQWSSLRASDTAMYYCARHTYPGPNSGYDYFEYWGQGTLVTVSS

SEQ ID NO: 48 - protein, 107 aa, Homo sapiens
TQSPSSLSASVGDRVNITCRASQSITNYLNWYQQKPGKAPRLLIYAASSLQSGVPSRFSGSGSGTDFTLTISSLQPEDFATYFCQQSYSNRWTFGQGTKLEIKRTVA

SEQ ID NO: 49 - protein, 104 aa, Homo sapiens; identical to SEQ ID NO: 10

(19) United States
(12) Patent Application Publication
(10) Pub. No.: US 2011/0171233 A1
(43) Pub. Date: Jul. 14, 2011

(54) Antibodies Against Human Cytomegalovirus (HCMV)

(75) Inventors: Ada Funaro, Torino (IT); Giorgio Gribaudo, Villafalletto (IT); Santo Landolfo, Torino (IT)

(73) Assignee: RIBOVAX BIOTECHNOLOGIES S.A., Petit-Lancy (CH)

(21) Appl. No.: 12/674,617

(22) PCT Filed: Jul. 31, 2008
(86) PCT No.: PCT/EP2008/060095

§ 371 Date: Mar. 22, 2011

(30) Foreign Application Priority Data
Aug. 22, 2007 (EP) 07114782.1
Aug. 31, 2007 (EP) 07115410.8

(51) Int. Cl. A61K 39/395; C07K 16/08; C07K 19/00; C07H 21/00; C07H 21/04; C12N 15/63; C12N 5/10; C12N 1/00; A61K 31/7088; C12P 21/02; C12Q 1/70; A61P 31/22

(52) U.S. Cl. 424/159.1; 530/324; 530/389.4; 536/23.53; 435/320.1; 435/339; 435/243; 514/44 R; 435/69.6; 435/5

(57) ABSTRACT

The present invention provides novel antibodies sequences that bind human cytomegalovirus (hCMV) and neutralize hCMV infection. The novel sequences can be used for the medical management of hCMV infections, in particular for preparing pharmaceutical compositions to be used in the prophylactic or therapeutic treatment of hCMV infections.

### TECHNICAL FIELD

The invention relates to novel antibody sequences isolated from human B cells having biological activities specific for a virus that infects human cells.

### BACKGROUND OF THE INVENTION

Human Cytomegalovirus (hCMV) is a widespread, highly species-specific herpesvirus, causing significant morbidity and mortality in immunosuppressed or immunologically immature individuals.

Several recent reviews analyze hCMV biology and clinical manifestations (Landolfo S et al., 2003; Gandhi M and Khanna R, 2004; Soderberg-Naucler C, 2006a). This viral pathogen infects the majority of the population worldwide and is acquired in childhood, following the contact with a bodily fluid, since the virus enters through human endothelial cells and epithelial cells of the upper alimentary or respiratory systems, or through the genitourinary system. Seropositivity to hCMV is more prevalent in underdeveloped countries or in geographical areas with lower income.

Following a primary infection, hCMV can persist in specific host cells of the myeloid lineage in a latent state, replicating and disseminating in many different cell types (haematopoietic cells, epithelial cells, endothelial cells, or fibroblasts) and escaping the host immune system. In fact, even though hCMV infections are maintained under control by the immune system, total hCMV clearance is rarely achieved.

The immunocompetent host can reduce the dissemination of the virus, in particular by means of humoral immunity, but hCMV has developed mechanisms that allow the viral genome to remain in selected sites in a latent state. In fact, any situation that weakens host immune functions, such as stress conditions, can lead to hCMV reactivation within hCMV-infected cells.

Clinical manifestations of hCMV (such as retinitis, enterocolitis, gastritis, or hepatitis) can be seen following primary infection, reinfection, or reactivation. Moreover, about 10% of infants are infected by the age of 6 months following transmission from their mothers via the placenta, during delivery, or by breastfeeding.

hCMV is a virus that has a linear, 230 kb, double-stranded DNA genome. The expression of the hCMV genome is controlled by a cascade of transcriptional events that leads to the synthesis of more than 200 proteins that perform a large variety of biological activities (Britt W and Mach M, 1996). The structural proteins form the virion envelope that is extremely complex and still incompletely defined. It includes glycoproteins that are homologues to structural proteins identified in other herpesviridae (gB, gH, gL, gM, and gN) and can form disulfide-linked protein complexes within the virion: gCI (including only gB), gCII (including gM and gN) and gCIII (including gH, gL, and gO). The glycoproteins gN and gM are the most abundant and, together with gH and gB, have been shown to be essential for initial interaction between the envelope of the infectious virion and the host cell, and consequently the production of infectious hCMV. For this reason, compounds targeting gB, gH, gN, or gM inhibit hCMV infection by blocking the entry of circulating hCMV virions into cells.

Treatment of hCMV infections is difficult because there are few options. The presently available drugs that inhibit viral replication (Ganciclovir, Cidofivir, Foscarnet, Maribavir, and others drugs under development) produce a significant clinical improvement, but may suffer from poor oral bioavailability, low potency, the emergence of hCMV resistance (due to mutations in the viral targets), and dose-limiting toxicities (De Clercq E, 2003; Baldanti F and Gerna G, 2004; Gilbert C and Boivin G, 2005).

Novel means for preventing and treating hCMV infection are needed, especially for immunocompromised individuals, in transplantation settings, and in prenatal prevention. In fact, hCMV is a clinically important opportunistic pathogen in HIV patients and in organ transplant recipients, where it contributes to graft loss independently from graft rejection, resulting in morbidity and mortality. The rising number of bone marrow and solid organ-transplant recipients raises the likelihood of hCMV clinical manifestations, such as hCMV pneumonitis or retinitis, in HIV-negative patients (Wiegland T and Young L, 2006). Moreover, hCMV is the major infectious cause of birth defects (such as hearing loss, delayed development, or mental retardation) which is due to a congenital or perinatal hCMV infection transmitted by an hCMV-infected mother (Griffiths P and Walter S, 2005).

Thus, it is important to provide drugs for universal preemptive, prophylactic hCMV-specific treatments, for example for the prevention of hCMV disease in transplant recipients (Hebart H and Einsele H, 2004; Kalil A et al., 2005; Snydman D, 2006), in patients developing hCMV-related neuropathologies (Griffiths P, 2004) or in connection to pregnancy (Schleiss M, 2003), to prevent the vertical transmission and life-threatening hCMV infection to foetuses and neonates.

Pharmaceutical compositions against hCMV may be useful for the treatment of other, more widespread diseases (such as cardiovascular and autoimmune diseases, or some types of cancer). In fact, hCMV is considered as a possible cofactor for such diseases and is associated to mechanisms leading to cell apoptosis, differentiation, and migration. Thus, hCMV is now a human pathogen of growing importance, for example, for long-term complications in tumour invasiveness and immune evasion, and for autoimmune or vascular diseases such as atherosclerosis or restenosis, wherein hCMV infection may alter cellular and immunological functions (Cinatl J et al., 2004; Soderberg-Naucler C, 2006b).

An alternative way to prevent hCMV infection may be vaccination, which would provide protection, at least in an array of high-risk patient populations. However, the correlation between vaccination and the resulting immune response is not fully clarified and an optimal hCMV vaccine strategy (using specific candidate antigens or live attenuated vaccines) depends on the patient population being targeted for protection. Therefore, prophylactic vaccination strategies are still under evaluation or have already failed in clinical settings (Schleiss M, 2005).

In view of the present limitations of pharmacological strategies for hCMV infections, the increasing knowledge of the host-hCMV relationship, and in particular, of the hCMV-specific immune response, makes immune-based therapies good candidates to substitute, or complement, existing treatments for the successful treatment of hCMV-associated complications (Gandhi M and Khanna R, 2004).

A possible alternative can be passive immunotherapy, consisting in the administration to individuals of pharmaceutical compositions comprising therapeutic antibodies with a defined binding specificity for a pathogenic antigen (e.g. hCMV).

This therapeutic approach has been built on the antigen-binding features of antibodies and antibody fragments directed against human or non-human therapeutic targets (Dunman P and Nesin M, 2003; Keller M and Stiehm E, 2000). Passive immunotherapy has been introduced into clinical practice, rapidly expanding the opportunities for the treatment of a wide variety of diseases (including infectious diseases, immune-mediated diseases, and cancer). This approach can be particularly effective in patients whose immune system is unable to produce antibodies in the amounts and/or with the specificity required to block and/or eliminate the targeted molecule (Chatenoud L, 2005; Laffly E and Sodoyer R, 2005).

In the field of hCMV treatment, a similar approach is now performed by administering intravenously human immunoglobulin preparations that are obtained by pooling human plasma with high titers of anti-hCMV antibodies, and commercialized for clinical uses (under the name of Cytotect or CytoGam). However, such a therapeutic approach represents only a partially satisfactory solution for blocking hCMV infection, in particular in immuno compromised patients where potent antivirals are often co-administered (Bonaros N et al., 2004; Kocher A et al., 2003; Kruger R et al., 2003).

Human purified, recombinant antibodies that have high affinity for antigens on hCMV surface would obviously represent much better drugs for passive immunization. In fact, several of the hCMV glycoproteins elicit strong host immune responses, including the production of virus-neutralizing antibodies, even though the stoichiometry of the envelope proteins is variable and may be altered to escape host immune response. This response is considered to be a key component of host immunity and represents a goal of both antibody and vaccine development.

The hCMV envelope glycoproteins B (gB) and H (gH) are the molecular targets for hCMV-neutralizing antibodies for which more detailed information is available. Sera from seropositive individuals as well as monoclonal antibodies directed against these glycoproteins inhibit hCMV infection of cell cultures in vitro. In fact, there is a correlation between anti-gB and anti-gH titers and overall neutralizing activity of convalescent sera, and a significant drop of the sera neutralizing capacity after adsorption of gB- and gH-specific antibodies. Thus, hCMV envelope glycoproteins gB and gH contain antigenic domains that induce neutralizing antibodies (Britt W and Mach M, 1996; Landolfo S et al., 2003; Schrader J and McLean G, 2007).

Human monoclonal antibodies are the most preferable molecules for clinical applications, due to the poor results obtained with murine monoclonal antibodies. However, the development of such human antibodies for hCMV treatment has been interrupted since no virological or clinical benefits were observed in studies that evaluated the efficacy of monoclonal antibodies, for example, in hematopoietic stem cell transplantation (Boeckh M et al., 2001), or in retinitis (Gilpin A et al., 2003).

Failure of different antibodies to demonstrate clinical benefits in large trials warrants further studies aimed at the selection of antibodies, in particular fully human monoclonal antibodies that more efficiently neutralize the widest variety of hCMV clinical isolates. The treatment of CMV infections would benefit from having more potent pharmaceutical compositions comprising purified human monoclonal antibodies obtained from human cells maintained in cell culture conditions or, as recombinant proteins, from the expression of human genes coding for such antibodies in mammalian cells approved for regulatory purposes.

### DISCLOSURE OF THE INVENTION

The present invention provides novel antibody sequences that bind and neutralize hCMV, and that can be used for detecting, treating, inhibiting, preventing, and/or ameliorating hCMV infection or an hCMV-related disease.

Human B cells were obtained from an hCMV-seropositive individual and immortalized. This polyclonal population of immortalized, human B cells were used for generating subcultures that were tested for the presence of IgG antibodies in the cell culture supernatant neutralizing hCMV infection in vitro. Among the selected subcultures, the neutralizing activity, the isotype, and the clonality were determined for the antibodies secreted by the subcultures named 8C10, 10B7 and 8A11. These antibodies recognize the hCMV envelope glycoprotein B (gB), which is known to be the molecular target of antibodies neutralizing ability confirmed using in vitro models for hCMV infection.

The DNA sequences that encode the variable regions of the antibody secreted by the 8C10, 10B7 and 8A11 subcultures were amplified, cloned, and sequenced. The corresponding protein sequences were analyzed to identify the Complementarity Determining Regions (CDRs) that are responsible for the hCMV-specific biological activity. These sequences can be used for producing proteins having hCMV-specific binding and neutralizing properties, in the form of full antibodies, antibody fragments, or any other format of functional protein (e.g. bioactive peptide, fusion proteins) using the appropriate technologies for producing recombinant proteins.

Compositions having therapeutic, prophylactic, and/or diagnostic utility in the management of hCMV infection and hCMV-related disorders can be prepared using these recombinant proteins, or antibodies purified from cell cultures originated from the 8C10, 10B7, 37B7 or 8A11 subcultures.

Further embodiments of the present invention will be provided in the following Detailed Description.

### DESCRIPTION OF THE FIGURES

FIG. 1: (A) Schematic representation of the CG3 antigen that has been assembled and used in ELISA as described in the literature (Rothe M et al., 2001). The recombinant autologous interstrain fusion antigen CG3 corresponds to a combination of the gB Antigenic Domain 2 (AD2) from hCMV strains AD169 (SwissProt Acc. No. P06473) and Towne (SwissProt Acc. No. P13201; ATCC Cat. No. VR-977). The AD2 region contains a site (amino acids 70-81, underlined) that is conserved in different viral strains and that has been shown to be recognized by neutralizing antibodies (Qadri I et al., 1992; Kropff P et al., 1993). (B) Schematic representation of the gH Antigen included in the gH(Ag)-GST fusion protein used for the gH-based ELISA assay. The recombinant antigen gH(Ag)-GST corresponds to an in-frame fusion between the gH amino terminal region (amino acids 16-144) from the hCMV strain VR1814 (Revello M et al., 2001) and Glutathione-S-Transferase (GST). The amino terminus of gH contains a linear antibody binding site between residues 34-43 (underlined) that is recognized by neutralizing antibodies (Urban M et al., 1992).

FIG. 2: overview of the selection process for identifying and characterizing subcultures (wells) that contain IgG antibodies binding and neutralizing hCMV. The subcultures were obtained by immortalizing B cells from CMV5 donor using the process described in WO 07/068758 and in the patent application EP07110693.4, by seeding 20 cells/well as a single subcloning step.

FIG. 3: overview of the selection process for identifying and characterizing subcultures (wells) that contain IgG antibodies binding and neutralizing hCMV. The subcultures were obtained by immortalizing B cells from CMV7 patient using the process described in WO 07/068758 and in the patent application EP07110693.4, by seeding 20 cells/well as a single subcloning step.

FIG. 4: gB-specific binding activity of IgG-containing supernatants from subcultures of immortalized human B cells against the fusion antigen CG3 that contains the gB (AD2) sequences (see FIG. 1). The ELISA was performed using the cell culture medium only (medium, negative control), or the supernatant from subcultures 26A1 (described in the patent application EP07110693.4), 1F7 (described in the patent application EP07111741.0), 8C10 and 37B7. The dotted line represents the threshold value (O.D 0.1) for considering a subculture positive.

FIG. 5: gB-specific binding activity of IgG-containing supernatants from subcultures of immortalized human B cells against the fusion antigen CG3 that contains the gB (AD2) sequences (see FIG. 1). The ELISA was performed using the cell culture medium only (medium, negative control), or the supernatant from subcultures 26A1 (described in the patent application EP07110693.4), 1F7 (described in the patent application EP07111741.0), 8A11, and 10B7. The dotted line represents the threshold value (O.D 0.1) for considering a subculture positive.

FIG. 6: hCMV neutralizing activity for the 8C10 antibody, as purified by affinity chromatography from the supernatant of a 8C10 subculture-derived cell culture maintained in serum-free medium. The dose-response analysis was performed in the hCMV assay including either human embryonic fibroblast (HELF) cells together with the hCMV strain AD169 (1,000 PFU/reaction; IC50 0.82 μg/ml; square), or human umbilical vein endothelial cells (HUVEC) cells together with the hCMV strain VR1814 (1,000 PFU/reaction; IC50 0.67 μg/ml; triangle).

FIG. 7: (A) Alignment of the DNA (lower case, 396 base pairs) and protein (upper case, 132 amino acids) consensus sequence of the variable region for the heavy chain of the 8C10 antibody (VH 8C10; SEQ ID NO.: 4 and 5). (B) Protein consensus sequence for VH 8C10 with the indication of predicted CDRs of VH 8C10 (HCDR1, HCDR2, and HCDR3; underlined; SEQ ID NO.: 6, 7, and 8). Alternative amino acids that were encoded by the DNA sequences cloned in plasmid from isolated E. coli transformants are indicated below the consensus protein sequence.

FIG. 8: (A) Alignment of the DNA (lower case, 312 base pairs) and protein (upper case, 104 amino acids) consensus sequence of the variable region for the light chain of the 8C10 antibody (VL 8C10; SEQ ID NO.: 9 and 10). (B) Protein consensus sequence for VL 8C10 with the indication of predicted CDRs of VL 8C10 (LCDR1, LCDR2, and LCDR3; underlined; SEQ ID NO.: 11, 12, and 13). Alternative amino acids that were encoded by the DNA sequences cloned in plasmids from isolated E. coli transformants are indicated below the consensus protein sequence.

FIG. 9: (A) Alignment of the DNA (lower case, 357 base pairs) and protein (upper case, 119 amino acids) consensus sequence of the variable region for the heavy chain of the 37B7 antibody (VH 37B7; SEQ ID NO.: 14 and 15). (B) Protein consensus sequence for VH 37B7 with the indication of predicted CDRs of VH 37B7 (HCDR1, HCDR2, and HCDR3; underlined; SEQ ID NO.: 16, 17, and 18). Alternative amino acids that were encoded by the DNA sequences cloned in plasmids from isolated E. coli transformants are indicated below the consensus protein sequence.

FIG. 10: (A) Alignment of the DNA (lower case, 321 base pairs) and protein (upper case, 107 amino acids) consensus sequence of the variable region for the light chain of the 37B7 antibody (VL 37B7; SEQ ID NO.: 19 and 20). (B) Protein consensus sequence for VL 37B7 with the indication of predicted CDRs of VL 37B7 (LCDR1, LCDR2, and LCDR3; underlined; SEQ ID NO.: 21, 22, and 23). Alternative amino acids that were encoded by the DNA sequences cloned in plasmids from isolated E. coli transformants are indicated below the consensus protein sequence.

FIG. 11: (A) Alignment of the protein consensus sequence of the variable region for the heavy chain (VH) of antibody sequences identified in immortalized B cells isolated from CMV7 patient and disclosed in the present patent application (VH 8C10 SEQ ID NO.: 5; VH 37B7, SEQ ID NO.: 15), and in the patent application EP07110693.4 (VH 26A1, SEQ ID NO.: 24). (B) Alignment of the protein consensus sequence of the variable region for the light chain (VH) of antibody sequences identified in immortalized B cells isolated from CMV7 patient and disclosed in the present patent application (VL 8C10, SEQ ID NO.: 10, VL 37B7, SEQ ID NO.: 20), and in EP07110693.4 (VL 26A1; SEQ ID NO.: 25).

FIG. 12: (A) Alignment of the DNA (lower case, 372 base pairs) and protein (upper case, 124 amino acids) consensus sequence of the variable region for the heavy chain of the 10B7 antibody (VH 10B7; SEQ ID NO.: 26 and 27). (B) Protein consensus sequence for VH 10B7 with the indication of predicted CDRs of VH 10B7 (HCDR1, HCDR2, and HCDR3; underlined; SEQ ID NO.: 28, 29, and 30). Alternative amino acids that were encoded by the DNA sequences cloned in plasmids from isolated E. coli transformants are indicated below the consensus protein sequence.

FIG. 13: (A) Alignment of the DNA (lower case, 336 base pairs) and protein (upper case, 112 amino acids) consensus sequence of the variable region for the light chain of the 10B7 antibody (VL 10B7; SEQ ID NO.: 31 and 32). (B) Protein consensus sequence for VL 10B7 with the indication of predicted CDRs of VL 10B7 (LCDR1, LCDR2, and LCDR3; underlined; SEQ ID NO.: 33, 34, and 35).

FIG. 14: (A) Alignment of the DNA (lower case, 384 base pairs) and protein (upper case, 128 amino acids) consensus sequence of the variable region for the heavy chain (VH) of the 8A11 antibody (VH 8A11; SEQ ID NO.: 36 and 37). (B) Protein consensus sequence for VH 8A11 with the indication of predicted CDRs of VH 8A11 (HCDR1, HCDR2, and HCDR3; underlined; SEQ ID NO.: 28, 39, and 40). Alternative amino acids that were encoded by the DNA sequences cloned in plasmids from isolated E. coli transformants are indicated below the consensus protein sequence.

FIG. 15: (A) Alignment of the DNA (lower case, 336 base pairs) and protein (upper case, 112 amino acids) consensus sequence of the variable region for the light chain (VL) of the 8A11 antibody (VL 8A11; SEQ ID NO.: 41 and 42). (B) Protein consensus sequence for VL 8A11 with the indication of predicted CDRs of VL 8A11 (LCDR1, LCDR2, and LCDR3; underlined; SEQ ID NO.: 43, 44, and 45).

FIG. 16: (A) Alignment of the protein consensus sequence of the variable region for the heavy chain (VH) of antibody sequences identified in immortalized B cells isolated from CMV5 donor and disclosed in the present patent application (VH 10B7 SEQ ID NO.: 27; VH 8A11, SEQ ID NO.: 37), in the patent application EP07111741.0 (VH 1F7, SEQ ID NO.: 46), and in WO 07/068758 (VH 9G8; SEQ ID NO.: 47). (B) Alignment of the protein consensus sequence of the variable region for the heavy chain (VH) of antibody sequences identified in immortalized B cells isolated from CMV5 donor and disclosed in the present patent application (VL 10B7, SEQ ID NO.: 32, VL 8A11, SEQ ID NO.: 42), in the patent application EP07111741.0 (VL 1F7, SEQ ID NO.: 26), and in WO 07/068758 (VL 9G8; SEQ ID NO.: 49).

### DETAILED DESCRIPTION OF THE INVENTION

The methods described in WO 07/068758 allow the efficient immortalization of isotype-specific human B cells obtained from an individual, whose blood contains antibodies having biological activities of interest (e.g. neutralizing a viral target).

In the present case, IgG-secreting cell cultures of immortalized human B cells were obtained from the blood of an hCMV patient in which an hCMV-neutralizing activity was initially detected. This biological activity was then used to select subcultures of immortalized B cells obtained from the original polyclonal population of human EBV-immortalized B cells.

The present invention provides novel protein sequences that are capable of binding and neutralizing hCMV. These protein sequences include specific CDRs (Complementarity Determining Regions) identified in the variable regions of the heavy and light chains of the antibody expressed by the 8C10, 37B7, 10B7 and 8A11 subcultures, and that can be briefly indicated as the 8C10, 37B7, 8A11 and the 10B7 antibody, respectively.

The Examples show the specificity of 8C10, 37B7, 10B7 and 8A11 antibodies for an immunodominant region of the hCMV glycoprotein B (gB; also known as also UL55 or gpUL55). This glycoprotein consists of a 110-116 kD ectodomain linked by disulfide bonds to a 55 kD transmembrane component, which then is linked to another unit forming the mature homodimer. This glycoprotein can contain up to 50-60 kD of N-linked sugars and it can be also phosphorylated (Britt W and Mach M, 1996). Glycoprotein B is considered as one of the major glycoprotein of the envelope of this virus, and four genotypes of hCMV that are detected in hCMV patients can be classified on the basis of the sequence variation in the UL55 gene that encodes gB (Coaquette A et al., 2004; Humar A et al., 2003).

Detailed maps of gB antigenic and functional protein domains were determined using in vitro assays based on gB fragments and human sera and/or murine monoclonal antibodies, showing that gB gives rise to hCMV-neutralizing antibodies in most infected individuals. For example, hCMV-gB interaction has been studied using human sera from patients at different phase of infection (Alberola J et al., 2000). In particular, two major sites on gB are capable of eliciting potent neutralizing antibodies, and then are called Antigenic Domains 1 and 2 (AD-1 and AD-2; Qadri I et al., 1992; Marshall, G et al., 2000; Schrader J and McLean G, 2007). In particular, AD-2 appears as eliciting potent hCMV-neutralizing antibodies in humans, where the immune response to this antigen has been studied (Navarro D et al., 1997; Lantto J et al., 2002a; Lantto J et al., 2002b; Lantto J et al., 2003; Gicklhorn, D et al., 2003; McLean G et al., 2006).

A number of gB-specific murine, chimeric or humanized monoclonal antibodies have been generated using different technologies and characterized as having hCMV neutralizing capacity, thus suggesting their utility for the prophylaxis or treatment of hCMV infection (EP609580, EP248909, WO 91/15586, WO 93/21952, WO 98/02746, WO 07/094423). In particular, a human IgG1 monoclonal antibody called regavirumab (also known as monoclonal antibody C23 or MCA C23) was isolated from hCMV-stimulated human splenic lymphocytes that have been fused with mouse myeloma cells to generate hCMV-specific hetero-hybridomas (Matsumoto Y et al., 1986; Arizono H et al., 1994). This antibody, as the others, has not been further developed for clinical use, but fully human, recombinant monoclonal antibodies directed against gB that are endowed with a potent neutralizing activity against clinical strains of hCMV are still of interest for the prevention or the therapy of hCMV infection.

In one embodiment, the present invention provides proteins comprising a sequence having at least 90% identity with the sequence of the HCDR3 (CDR3 of the heavy chain variable region) of the 8C10 (SEQ ID NO.: 8) or 10B7 (SEQ ID NO.: 30) or the 8A11 antibody (SEQ ID NO.: 37) or the 37B7 antibody. The level of identity should be determined on the full length of such sequence.

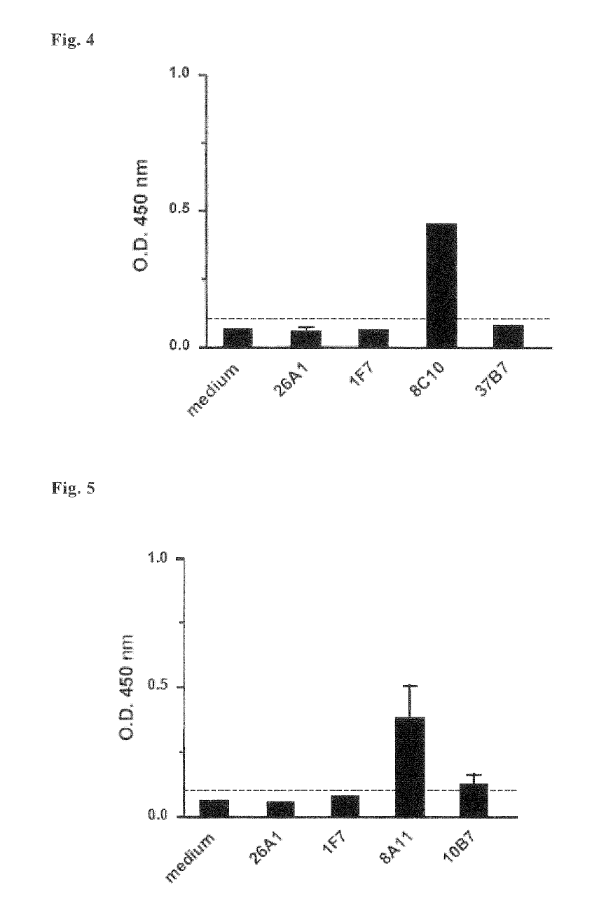

In the case of the 10B7 antibody, the HCDR3 is included, together with the HCDR1 and HCDR2 (SEQ ID NO.: 28 and SEQ ID NO.:29), this HCDR3 is included in the variable region of the heavy chain of the 10B7 antibody (VH 10B7; FIG. 4; SEQ ID NO.: 27.) This latter sequence is encoded by the DNA sequence (FIG. 4A; SEQ ID NO.: 26) that was amplified and cloned using the immortalized B cells from the original subculture secreting the antibody. Thus a protein of the invention may contain, together with the HCDR3 of the antibody, the sequence of the HCDR1 and/or HCDR2 of the 10B7 antibody. Such a protein may then comprise a sequence having at least 90% identity with the entire sequence of the variable region of the heavy chain of the 10B7 antibody.

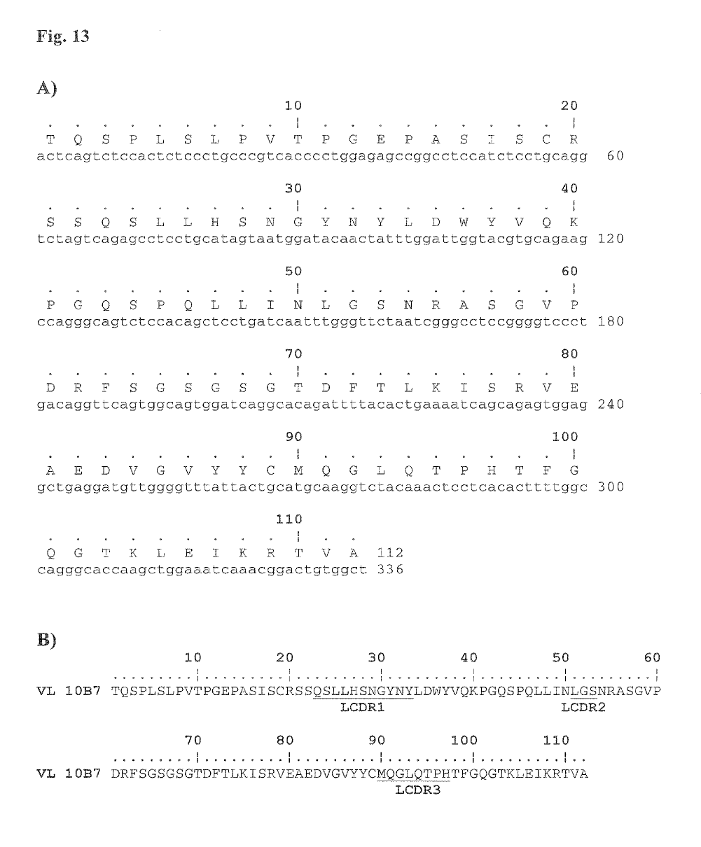

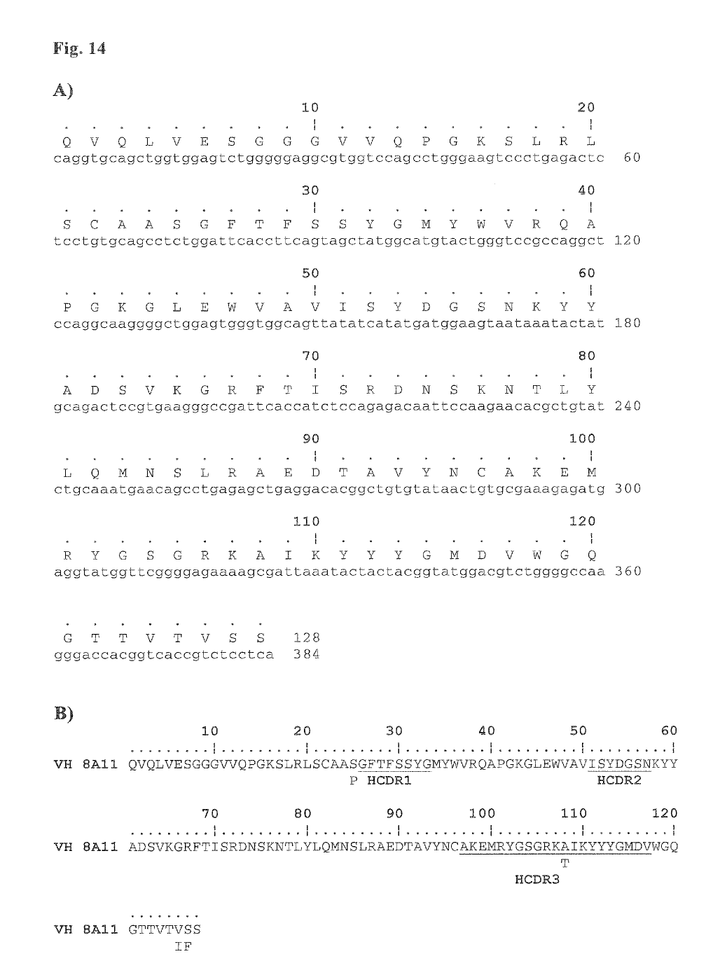

The 10B7 antibody also contains a variable region of a light chain for which, using the same approach, the DNA (SEQ ID NO.: 31) and the protein (SEQ ID NO.: 32) sequences, together with the specific LCDRs (SEQ ID NO.: 33, SEQ ID NO.: 34 and SEQ ID NO.: 35), have been determined (FIG. 13). Thus a protein of the Invention can further comprise one or more sequences selected from the group consisting of single LCDRs of the 10B7 antibody, which can be provided as a protein sequence comprising a sequence having at least 90% identity with VL 10B7. This applies in particular when a human recombinant antibody, comprising both the original VL 10B7 and VH 10B7 sequence, is desired.

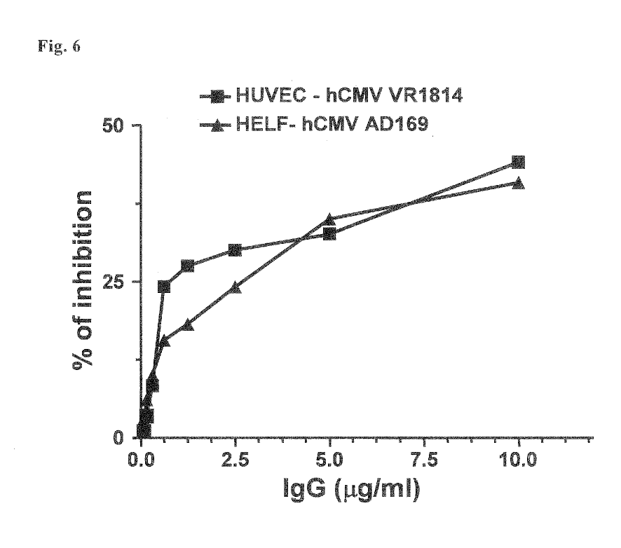

In the case of the 8A11 antibody, the HCDR3 is included, together with the HCDR1 and HCDR2 (SEQ ID NO.: 16 and SEQ ID NO.: 17), in the variable region of the heavy chain of the 8A11 antibody (VH 8A11; FIG. 6; SEQ ID NO.: 15.) This latter sequence is encoded by the DNA sequence (FIG. 6A; SEQ ID NO.: 14) that was amplified and cloned using the immortalized B cells from the original subculture secreting the 8A11 antibody. Thus a protein of the invention may contain, together with the HCDR3 of the 8A11 antibody, the sequence of the HCDR1 and/or HCDR2 of the 8A11 antibody. Such a protein may then comprise a sequence having at least 90% identity with the entire sequence of the variable region of the heavy chain of the 8A11 antibody.

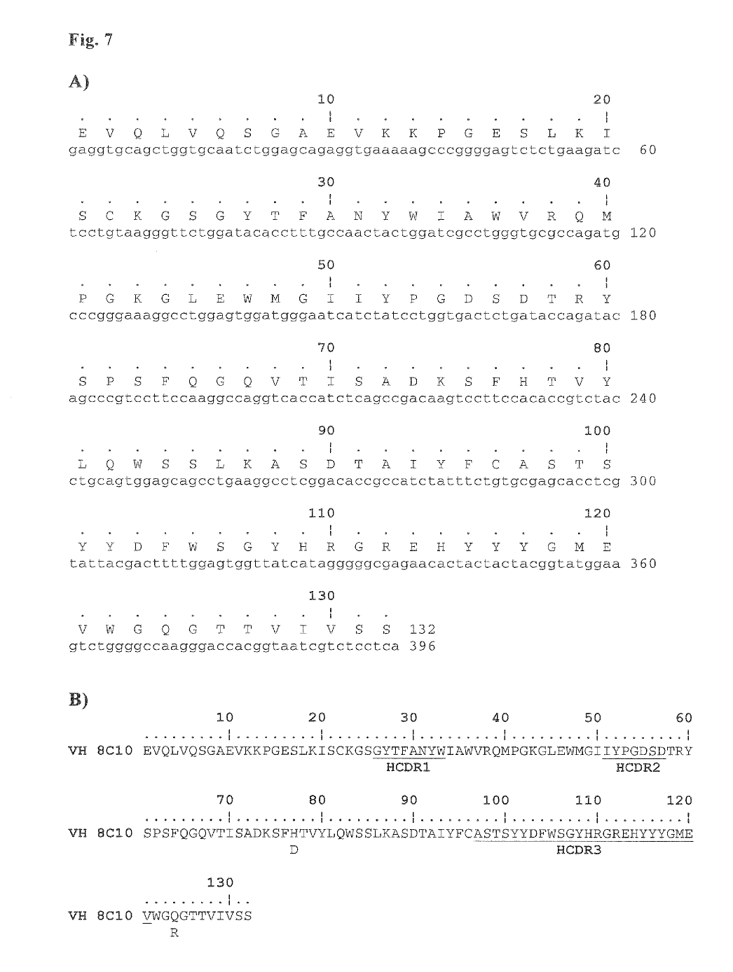

The 8A11 antibody also contains a variable region of a light chain for which, using the same approach, the DNA (SEQ ID NO.: 19) and the protein (SEQ ID NO.: 20) sequences, together with the specific LCDRs (SEQ ID NO.: 21, SEQ ID NO.: 22 and SEQ ID NO.: 23), have been determined (FIG. 7). Thus a protein of the Invention can further comprise one or more sequences selected from the group consisting of single LCDRs of the 8A11 antibody, which can be provided as a protein sequence comprising a sequence having at least 90% identity with VL 8A11. This applies in particular when a human recombinant antibody, comprising both the original VL 8A11 and VH 8A11 sequence, is desired.

The HCDR3 of the 10B7 and 8A11 antibodies can be considered as characterizing the antigen-binding portion of a specific human antibody that is capable of binding and neutralizing hCMV, as shown in the Examples. Even though, several or all CDRs of an antibody are generally required for obtaining an optimal antigen-binding surface, HCDR3 is the CDR showing the highest differences between antibodies not only with respect to sequence but also with respect to length.

Such diversities are fundamental components of binding regions for the recognition of essentially any antigen by the humoral immune system and HCDR3 can be sufficient for determining antigen specificity (Xu and Davis, 2000; Barrios Y et al. 2004; Bond C et al., 2003). Alternatively, combinations of CDRs can be linked to each other in very short proteins that retain the original binding properties, as recently reviewed (Ladner R, 2007).

Thus, hCMV-neutralizing proteins can be generated using the HCDR3 (or any other CDR) of 8C10, 10B7 or 8A11 antibodies as an hCMV binding moiety, in combination or not with other CDRs from the respective original antibody, which is expressed within an antibody protein framework (Knappik A et al., 2000), or a protein framework unrelated to antibodies (Kiss C et al., 2006).

The variable regions of the heavy and light chains forming 8C10, 8A11 or 10B7 antibodies (or selected portions, such as the isolated HCDRs and LCDRs) can be included in any other protein format for functional antibody fragments, as described in the literature under different names such as Scfv (single-chain fragment variable), Fab (variable heavy/light chain heterodimer), diabody, isolated heavy or light chains, bispecific antibodies, and the other engineered antibody variants reviewed in the literature (Laffly E and Sodoyer R, 2005; Jain M et al., 2007).

Alternative antibodies can be generated using the sequences of 8C10, 10B7 or 8A11 antibodies through a process of light-chain variable domain (VL) shuffling. In fact, several different human antibodies can be generated and tested for hCMV-specific activity using a single heavy-chain variable domain VH (such as the one of 8C10, 10B7 or 8A11 antibody) combined with a library of VL domains, at the scope of determining VH/VL combinations with improved properties in terms of affinity, stability, and/or recombinant production (Ohlin M et al., 1996; Rojas G et al., 2004; Watkins N et al., 2004).

Moreover, novel approaches for developing new bioactive peptides also showed the feasibility of synthesizing CDR-derived peptides that contain L-amino acids and/or D-amino acids, that maintain the original activity, and that may have a good pharmacological profile (Smith J et al., 1995; Levi M et al., 2000; Wijkhuisen A et al., 2003). Thus, the HCDR3 of the 8C10, 10B7 or 8A11 antibody, as well as sequences highly similar to HCDR3 of 8C10, 10B7, 37B7 or 8A11 antibody, fusion proteins containing it, and synthetic peptides derived from them (e.g. containing L-amino acids and/or D-amino acids, in the normal or in the retro-inverse conformation), can be tested and used as hCMV-binding and neutralizing proteins.

Moreover, it is known that antibodies may be modified in specific positions in order to have antibodies with improved features, in particular for clinical applications (such as better pharmacokinetic profile or higher affinity for an antigen). These changes can be made in the CDRs and/or framework of the 8C10, 10B7, 37B7 or 8A11 antibody and the sequence can be chosen by applying any of the dedicated technologies for the rational design of antibodies that make use of affinity maturation and other processes (Kim S et al., 2006; Jain M et al., 2007).

The proteins of the invention may be provided as antibodies in general, fully human monoclonal antibodies having a specific isotype (e.g. IgG, that is the antibody format of almost all approved therapeutic antibodies) in particular, antibody fragments, bioactive peptides or fusion proteins. All these alternative molecules should maintain, if not enhance, the original hCMV binding and neutralization properties that were determined for the 8C10, 10B7, 37B7 and 8A11 antibodies.

In the case of fusion proteins, the heterologous protein sequences can be located in the N- or C-terminal position to the 8C10-, 10B7-, 37B7-, or 8A11-derived sequence, without affecting the correct expression and biological activity of the hCMV-specific moiety (e.g. an antibody fragment).

The term “heterologous protein” indicates that a protein sequence is not naturally present in the N- or C-terminal position to the hCMV-specific moiety (e.g. an antibody fragment). The DNA sequence encoding this protein sequence is generally fused by recombinant DNA technologies and comprises a sequence encoding at least 5 amino acids.

Such a heterologous protein sequence is generally chosen for providing additional properties to the hCMV-specific antibody fragment for specific diagnostic and/or therapeutic uses. Examples of such additional properties include: better means for detection or purification, additional binding moieties or biological ligands, or the post-translational modification of the fusion protein (e.g. phosphorylation, glycosylation, ubiquitination, SUMOylation, or endoproteolytic cleavage).

Alternatively (or additionally to the fusion to a heterologous protein sequence), the activity of a protein of the invention may be improved with the conjugation to different compounds such as therapeutic, stabilizing, or diagnostic agents. Examples of these agents are detectable labels (e.g. a radioisotope, a fluorescent compound, a toxin, a metal atom, a chemiluminescent compound, a bioluminescent compound, or an enzyme) that can be bound using chemical linkers or polymers. The hCMV-specific biological activity may be improved by the fusion with another therapeutic protein, such as a protein or a polymer altering the metabolism and/or the stability in diagnostic or therapeutic applications.

Means for choosing and designing protein moieties, ligands, and appropriate linkers, as well as methods and strategies for the construction, purification, detection and use of fusion proteins are provided in the literature (Nilsson et al., 1997; “Applications Of Chimeric Genes And Hybrid Proteins” Methods Enzymol. Vol. 326-328, Academic Press, 2000; WO01/77137) and are commonly available in clinical and research laboratories. For example, the fusion protein may contain sequences recognized by commercial antibodies (including tags such as polyhistidine, FLAG, c-Myc, or HA tags) that can facilitate the in vivo and/or in vitro identification of the fusion protein, or its purification.

Other protein sequences can be easily identified by direct fluorescence analysis (as in the case of Green Fluorescent Protein), or by specific substrates or enzymes (using proteolytic sites, for example). The stability of the hCMV-specific antibodies, antibody fragments, and fusion proteins may be improved with the fusion of well-known carrier proteins, such as phage coat protein (cp3 or cp8), Maltose Binding Protein (MBP), Bovine Serum Albumin (BSA), or Glutathione-S-Transferase (GST).

The 8C10, 10B7, 37B7 and 8A11 antibodies are the main objects of the invention and they have been characterized, using the specific subculture supernatant, as a human IgG1 antibody which are capable of neutralizing hCMV, as determined by in vitro neutralization assays (Table 1), and to bind to a region of the hCMV gB envelope glycoprotein (FIG. 3). Consequently, this IgG antibody can be used for defining other hCMV-neutralizing proteins (e.g. in form of the antibodies, antibody fragments, bioactive peptides, fusion protein, or any natural/recombinant proteins) that are capable of neutralizing hCMV infection by binding gB in this region or in specific epitopes contained within. These properties can be tested using the assay described in the examples, or in any other hCMV specific assay. Such competing proteins may contain (or not) the HCDR3 defined above, optionally together with HCDRs and LCDRs in part or completely identical from those originally identified in the 10B7 and 8A11 antibodies.

Further objects of the inventions are the nucleic acids encoding any of the antibodies, antibody fragments, fusion proteins, bioactive peptides, or isolated CDRs defined above.

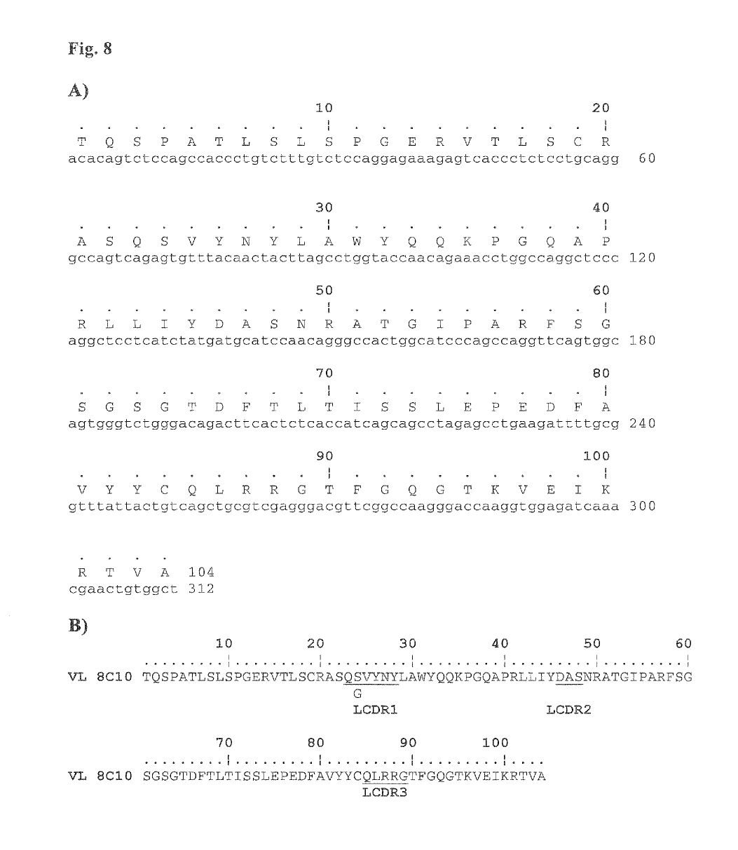

The examples provide such sequences in particular as encoding the full variable regions of the 8C10 heavy (SEQ ID NO.: 4) and light (SEQ ID NO.: 9) chains, or of the 8A11 heavy (SEQ ID NO.: 36) and light (SEQ ID NO.: 37) chains, or 10B7 heavy (SEQ ID NO.: 26) and light (SEQ ID NO.: 27) chains. These DNA sequences (or selected portions, such as those encoding the specific HCDRs and LCDRs; FIG. 8, 10, 15) can be transferred in vectors for expressing them in one of the alternative formats for antibodies (e.g. full, affinity-matured, CDR-grafted, or antibody fragments) or fusion proteins.

These nucleic acids can comprise a sequence having at least 90% identity with SEQ ID NO.: 4 (for 8C10-derived sequences) SEQ ID NO.: 31 (for 10B7-derived sequences) or SEQ ID NO.: 36 (for 8A11-derived sequences), with or without a sequence further comprising a sequence having at least 90% identity with SEQ ID NO.: 9 (for 8C10-derived sequences), SEQ ID NO.: 27 (for 10B7-derived sequences), SEQ ID NO.: 37 (for 8A11-derived sequences), respectively, depending on whether sequences from only the heavy chain of 8C10, 10B7 or 8A11 antibody, or from all the heavy and light chains of the 8C10, 10B7, 37B7 or 8A11 antibody are needed. When a fully human antibody is desirable, the antibody should further comprise a heavy chain constant region selected from the group consisting of human IgG1, IgG2, IgG3, IgG4, IgM, IgA and IgE constant regions.

The nucleic acid sequences encoding the full variable regions of 8C10, 10B7, 37B7 and 8A11 antibodies heavy and light chains have been cloned and characterized by means of PCR reactions and vectors transforming E coli cells. Such sequences can be transferred (in part or in their entirety) within other vectors, in particular into the expression cassette of a single vector or of distinct vectors where they are operably linked to the appropriate regulatory sequences (e.g. promoters, terminator of transcription).

The original 8C10, 10B7, 37B7 and 8A11 antibodies, or any other protein sequences derived from such antibody, can be expressed as recombinant proteins using such vectors for transforming the appropriate host cells.

The host cells comprising the nucleic acids of the invention can be prokaryotic or eukaryotic host cells and should allow the secretion of the desired recombinant protein. Methods for producing such proteins include culturing host cells transformed with the expression vectors comprising their coding sequences under conditions suitable for protein expression and recovering the protein from the host cell culture.

The nucleic acids and host cells can be used for producing a protein of the invention by applying common recombinant DNA technologies. Briefly, the desired DNA sequences can be either extracted by digesting the initial cloning vector with restriction enzymes, or amplified using such a vector as a template for a Polymerase Chain Reaction (PCR) and the PCR primers for specifically amplifying full variable regions of the heavy and light chains or only portions of them (e.g. the HCDR3 sequence). These DNA fragments can be then transferred into more appropriate vectors for expression into prokaryotic or eukaryotic host cells, as described in books and reviews on how to clone and produce recombinant proteins, including titles in the series “A Practical Approach” published by Oxford Univ. Press (“DNA Cloning 2: Expression Systems”, 1995; “DNA Cloning 4: Mammalian Systems”, 1996; “Protein Expression”, 1999; “Protein Purification Techniques”, 2001).

The vectors should include a promoter, a ribosome binding site (if needed), the start codon, and the leader/secretion sequence, that can drive accordingly the expression of a mono or bicistronic transcript having the DNA coding for the desired protein. The vectors should allow the expression of the recombinant protein in the prokaryotic or eukaryotic host cells. A cell line substantially enriched in such cells can be then isolated to provide a stable cell line.

For eukaryotic hosts (e.g. yeasts, insect or mammalian cells), different transcriptional and translational regulatory sequences may be employed, depending on the nature of the host. They may be derived from viral sources, such as adenovirus, bovine Papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for the transient (or constitutive) repression and activation and for modulating gene expression.

The sequence encoding the recombinant protein can be adapted and recloned for making modifications at the DNA level only that can be determined, for example, using software for selecting the DNA sequence in which the codon usage and the restriction sites are the most appropriate for cloning and in expression in specific vectors and the host cells (Grote A et al., 2005; Carton J et al., 2007).

During further cloning steps, protein sequences can be added in connection to the desired antibody format (Scfv, fab, antibody fragment, fully human antibody, etc.), or to the insertion, substitution, or elimination of one or more internal amino acids. These technologies can also be used for further structural and functional characterization and optimization of the therapeutic properties of proteins in general, and of antibodies in particular (Kim S et al., 2005), or for generating vectors allowing their stable in vivo delivery (Fang J et al., 2005). For example, recombinant antibodies can also be modified at the level of structure and/or activity by choosing a specific Fc region to be fused to the variable regions (Furebring C et al., 2002; Logtenberg T, 2007), by adding stabilizing peptide sequences, (WO 01/49713), by generating recombinant single chain antibody fragments (Gilliland L et al., 1996), or by adding radio chemicals or polymers to chemically modified residues (Chapman A et al., 1999).

The DNA sequence coding for the recombinant protein, once inserted into a suitable episomal or non-homologously or homologously integrating vector, can be introduced in the appropriate host cells by any suitable means (transformation, transfection, conjugation, protoplast fusion, electroporation, calcium phosphate precipitation, direct microinjection, etc.) to transform them. Important factors to be considered when selecting a particular vector include: the ease with which host cells that contain the vector may be recognized and selected; the number of copies of the vector which are desired; and whether the vector is able to “shuttle” the vector between host cells of different species.

The cells which have been stably transformed by the introduced DNA can be selected by also introducing one or more markers which allow for selection of host cells which contain the expression vector. The marker may also provide for phototrophy to an auxotropic host, biocide resistance, e.g. antibiotics, or heavy metals such as copper, or the like, and may be cleavable or repressed if needed. The selectable marker gene can either be directly linked to the DNA gene sequences to be expressed, or introduced into the same cell by co-transfection. Additional transcriptional regulatory elements may also be needed for optimal expression.

Host cells may be either prokaryotic or eukaryotic. Amongst prokaryotic host cells, the preferred ones are B. subtilis and E. coli. Amongst the eukaryotic host cells, the preferred ones are yeast, insect, or mammalian cells. In particular, cells such as human, monkey, mouse, insect (using baculovirus-based expression systems) and Chinese Hamster Ovary (CHO) cells, provide post-translational modifications to protein molecules, including correct folding or certain forms of glycosylation at correct sites. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognize leader sequences in cloned mammalian gene products and secrete peptides bearing leader sequences (i.e., pre-peptides).

Mammalian cell lines available as hosts for expression are known in the art and include many immortalized cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Per.C6, Bowes melanoma and human hepatocellular carcinoma (for example Hep G2) cells and a number of other cell lines. In the baculovirus system, the materials for baculovirus/insect cell expression systems are commercially available in kit form (e.g. commercialized by Invitrogen).

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1 or more days in an enriched media before they are switched to selective media. The purpose of the selectable marker is to confer resistance to selection, and its presence allows growth and recovery of cells that successfully express the introduced sequences. Resistant clones of stably transformed cells may proliferate using tissue culture techniques appropriate to the cell type. A cell line substantially enriched in such cells can be then isolated to provide a stable cell line.

In the case of full recombinant human immunoglobulins, an important step is the selection of the specific isotype and constant region. Vectors specifically designed for expressing antibodies with the desired isotype and subtype (for example, human IgG1, IgG2, or IgG4) are widely described in the literature. Then, the full antibodies or the fusion proteins can be expressed as recombinant proteins in prokaryotic organisms (e.g. Escherichia coli; Sorensen and Mortensen, 2005; Venturi et al., 2002), plants (Ma et al., 2005), or eukaryotic cells, that allow a high level of expression as transient or stable transformed cells (Dinnis D and James D, 2005). This would be required in particular when the characterization of the antibodies has to be performed using more sophisticated assays, including in vivo assays, where the half-life of the antibody can be determined. The host cells can be further selected on the basis of the expression level of the recombinant protein.

In addition, when the protein is expressed, especially as an antibody, in eukayotic host cells (mammalian cell lines, in particular), different vectors and expression systems have been designed for generating stable pools of transfected cell lines (Aldrich T et al., 2003; Bianchi A and McGrew J, 2003). High level, optimized, stable expression of recombinant antibodies has been achieved (Schlatter S et al., 2005), also due to optimization of cell culture conditions (Grunberg J et al., 2003; Yoon S et al., 2004) and by selecting or engineering clones with higher levels of antibody production and secretion (Bohm E et al., 2004; Butler M, 2005).

The antibody, the antibody fragments, the fusion proteins, and any other protein defined above as being capable of binding and neutralizing hCMV can be purified using the well-established technologies that allow the isolation of either non-/recombinant proteins from cell culture or from synthetic preparations. These technologies should provide a sufficient amount of protein (from the microgram to the milligram range) to perform a more extensive characterization and validation for hCMV-related prophylactic, diagnostic, and therapeutic uses.

To this purpose, the preparations of recombinant proteins can be tested in in vitro or in vivo assays (biochemical, tissue- or cell-based assays, disease models established in rodents or primates, biophysical methods for affinity measurements, epitope mapping, etc.), in particular using one or more of those disclosed in the Examples or in the literature for studying hCMV pathogenesis and immunobiology.

The mechanism and the efficacy of hCMV neutralization, in connection to the binding by the 8C10, 10B7, 37B7 and 8A11 antibodies, and the other proteins defined above, can be characterized using the cell and/or animal models available for gB, as shown in the literature using panels of human sera (Navarro D et al., 1997) or of murine monoclonal antibodies (Schoppel K et al., 1996), possibly in combination with ELISA or Western Blot using hCMV-specific truncated proteins or synthetic peptides (Greijer A et al., 1999; Ohlin M et al., 1993; Nejatollahi F et al., 2002). However, the strict species specificity of hCMV requires elaborated animal models for studying the properties of antiviral compounds (such as hCMV-neutralizing antibodies) and the importance of host/hCMV genotypes, for example for intrauterine hCMV infection (Revello M and Gerna G, 2004; Barry P et al., 2006).

The antibodies, as purified preparations from human B cell supernatants or expressed as recombinant proteins, can be further validated using organ- or cell-based in vitro assays known in the literature (Eggers M et al. 1998; Lam V et al., 2006; Reinhardt B et al., 2003; Forthal D et al., 2001; Goodrum F et al., 2002). Moreover, relevant pre-clinical tests can be made in CMV-infected animals, in particular in models where human host cells can be transplanted into immunocompromised rodents (Gosselin J et al., 2005; Thomsen M et al., 2005).

The purification of the recombinant proteins of the invention can be carried out by any of the conventional methods known for this purpose, i.e. any procedure involving extraction, precipitation, chromatography, or the like. In particular, methods for antibody purification can make use of immobilized gel matrices contained within a column (Nisnevitch M and Firer M, 2001; Huse K et al., 2002; Horenstein A et al., 2003), exploiting the strong affinity of antibodies for substrates such protein A, protein G, or synthetic substrates (Verdoliva A et al., 2002; Roque A et al., 2004), or for specific antigens or epitopes (Murray A et al., 2002; Jensen L et al., 2004). After washing, the protein is eluted from the gel by a change in pH or ionic strength. Alternatively, HPLC (High Performance Liquid Chromatography) can be used. The elution can be carried out using a water-acetonitrile-based solvent commonly employed for protein purification.

The antibody, the antibody fragments, the bioactive peptides, the fusion proteins, and any other compound defined above on the basis of 8C10, 10B7, 37B7 or 8A11 antibodies sequences can be used for detecting, treating, inhibiting, preventing, and/or ameliorating hCMV infection. To this purpose, such compounds can be used for preparing diagnostic, therapeutic, or prophylactic compositions for the management of hCMV infection. In particular such compounds can be used for preparing pharmaceutical compositions, together with any pharmaceutically acceptable vehicle or carrier, or further comprising any additional therapeutic or prophylactic agent, such as vaccines, immunomodulating or antiviral compounds. In the latter case, the literature provides some examples of such compounds acting on hCMV replication (Foscarnet, Vanganciclovir, Fomivirsen, or Ganciclovir) and already tested in humans (De Clercq E, 2003.)

These compositions may comprise a antibody, an antibody fragment, a bioactive peptide, a fusion protein, and any other compound defined above on the basis of the 8C10, 10B7, 37B7 or 8A11 antibody sequences, or all of them, or even another hCMV-neutralizing antibody characterized by a different epitope, such as the ones described in the literature or in the patent application EP07110693.4 and EP07111741.0 (26A1 and 1F7, respectively). In fact, it is described in the literature that, when two or more antibodies directed to viral or human target are combined in a pharmaceutical composition, the resulting composition may show an improved therapeutic efficacy due not only to an additive effect but also a synergic effect. (Logtenberg T, 2007)

The compositions comprising any of the proteins (e.g. antibodies, antibody fragment, fusion proteins, bioactive peptides) and of the nucleic acids defined above can be used and administered to an individual with a hCMV-related diagnostic, therapeutic, or prophylactic purpose. A method for treatment, prophylaxis, or diagnosis of hCMV, or of hCMV-related disease can comprise the administration of a protein or of a nucleic acid as above defined.

These compositions can be administered as means for hCMV-specific passive immunization which provide therapeutic compounds (in particular therapeutic antibodies or therapeutic antibodies fragments) that, by targeting hCMV virions, can inhibit the propagation of the virus in the treated patient, and potentially block the outbreak of a viral infection in the population.

Depending on the specific use, the composition should provide the compound to the human subject (in particular a pregnant woman or any other individual that is infected by hCMV or considered at risk for hCMV due to contact with an hCMV-infected individual) for a longer or shorter period of time. To this purpose, the composition can be administered, in single or multiple dosages and/or using appropriate devices, through different routes: intramuscularly, intravenously, subcutaneously, topically, mucosally, by a nebulizer or an inhaler, as eyedrops, in non-/biodegradable matrix materials or using particulate drug delivery systems. In particular, the composition may allow topical or ocular administration, that represent a useful approach given the presence of hCMV in mucosae and eye. Moreover, antibodies and antibody fragments can be effective when applied topically to wounds (Streit M et al., 2006), cornea (Brereton H et al., 2005) or vagina (Castle P et al., 2002).

A pharmaceutical composition should provide a therapeutically or prophylactically effective amount of the compound to the subject that allows the compound to exert its activity for a sufficient period of time. The desired effect is to improve the status of the hCMV patient by controlling hCMV infection, reactivation, and/or re-infection, and by reducing at least some of the clinical manifestations of hCMV infection, such as retinitis, pancreatitis, pneumonitis, etc. (Landolfo S et al., 2003). For example, the composition should be administered at an effective amount from about 0.005 to about 50 mg/kg/body weight, depending on the route of administration and the status of the individual.

In the case of compositions having diagnostic uses, the compound should be detected using technologies commonly established in the clinical and research laboratories for detecting virus in biological samples (e.g. ELISA or other serological assays), or, when administered to a subject in vivo, at least 1, 2, 5, 10, 24, or more hours after administration.

The detection of hCMV can be performed, using the proteins of the invention, in substitution or coupled to the known means and procedures that have been established for monitoring chronic or acute hCMV infection in populations of immunocompetent and immunocompromised hosts. These techniques showed a correlation between the data generated in vitro and the clinical status (Gilbert G, 2002; Gerna G and Lilleri D, 2006).

The clinical development and use should be based on the characterization of the antibody pharmacokinetics and pharmacodynamics (Lobo E et al., 2004), the data preclinical and clinical safety (Tabrizi M and Riskos L, 2007), and the compliancy to international requirements for the production and quality control of monoclonal antibodies for therapeutic and in vivo diagnostic use in humans (Harris R et al. 2004).

The proteins of the invention can also be used for the preparation of a composition for detecting, treating, inhibiting, preventing, and/or ameliorating other, more widespread diseases (such as cardiovascular and autoimmune diseases, or some types of cancer) that can be defined as hCMV-related diseases. In these conditions, hCMV is considered as a possible cofactor since it is well-known that this virus is associated with inflammatory processes (by stimulating the expression of Fc receptors, cell adhesion molecules, chemokines and cytokines) and with alterations to the antigen-presentation pathways (by inhibiting MHC class I and II expression) leading to cell apoptosis, differentiation, and migration, for example in blood vessels and in actively proliferating cells (Cinatl J et al., 2004; Soderberg-Naucler C, 2006b).

The invention will now be described by means of the following Examples, which should not be construed as in any way limiting the present invention.

### EXAMPLES

### Example 1

### Production of Cell Cultures Secreting Human Monoclonal Antibodies that Neutralize hCMV

### Materials & Methods

### Production of the Culture of Immortalized Human B Cells

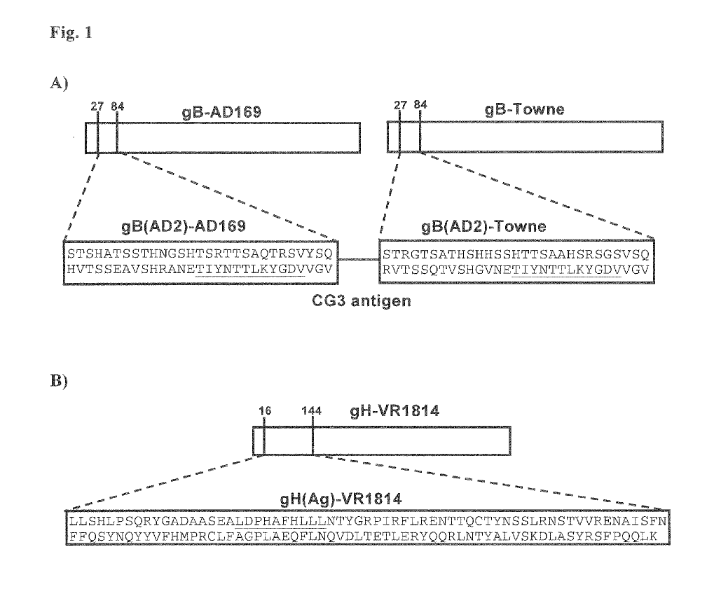

Peripheral blood mononuclear cells (PBMCs) were obtained from an asymptomatic blood donor with high hCMV-specific IgG titer in the serum (CMV5). The hCMV-neutralizing antibodies were detected in the serum according to an hCMV microneutralization assay based on human Embryo Lung Fibroblasts (HELF cells) and AD169 (an hCMV laboratory strain from ATCC, cod. VR-538). The serum was also tested in an ELISA specific for human IgG binding hCMV virion proteins that is commercially available (BEIA-CMV IgG Quant; Bouty, Cat. No. 21465) and in an ELISA specific for human IgG binding gB(AD2) hCMV (Biotest, Cat. No. 807035; Rothe M et al., 2001; FIG. 1A. These hCMV-specific assays have been performed as outlined in WO 07/068758 or as indicated by the Manufacturer.

The EBV immortalization process to which PBMCs from CMV5 were subsequently exposed has been described in WO 07/068758. At the end of the process, the immortalized cells were washed with fresh culture medium (RPMI-1640 added with 10% Fetal Calf Serum, FCS) and put in culture for 15 days at a density of 1.5×10⁶ cells/ml in 24-well plates with a feeder layer (irradiated PBMC seeded at 5×10⁵ cells/well). After this expansion phase, the hCMV neutralizing activity was confirmed with the test described above.

Selection of Subcultures of Immortalized Human B Cells that Secrete IgG Antibodies that Bind to Regions of the hCMV Envelope Glycoproteins gB and gH

Aliquots of the expanded cell culture (each statistically containing 20 cells) were seeded in to 96-well plates on irradiated, allogeneic PBMCs as feeder cells (50,000/well) in 100 μl IMDM (added with 10% FCS and Non Essential Amino Acids, NEAA, diluted 1× from a 100× commercial stock solution; EuroClone), with the addition of CpG2006 (1 μg/ml) and IL-2 (200 U/ml). A total of 3840 cultures were generated and, after two weeks, 50 μl of the same medium (including CpG2006 and IL-2 at the concentration indicated above) were added.

After a further 1-2 weeks, the supernatants of cell cultures that presented growing and aggregated cells were tested in parallel in ELISAs that detect binding of human IgG antibodies to regions of the gB or gH hCMV envelope glycoproteins (FIGS. 1A and 1B, respectively). In the case of the gB-GST antigen, the ELISA was performed by coating EIA polystyrene plates (Nunc; Cat No. 469949) with 100 ng/well of the bacterially expressed fusion protein between the gB immunodominant region from hCMV strain C194 and GST (Biodesign, Cat. No. R18102; GS-4B Sepharose Affinity purified, 1 mg/ml). The coating was performed overnight at 4° C., then, after blocking plates with PBS containing 1% of milk in each well for 1 hour at 37° C., 50 μl of supernatants from cell cultures were incubated in each well for 2 hours at 37° C. After four washings cycles, 50 μl of the secondary antibody (goat anti-human IgG (Fc specific) antibody conjugated with horseradish peroxidase; diluted 1:30000 in wash buffer; Sigma, Cat. No. A0170) was dispensed in each well and plates were incubated for 1 hour at room temperature. After four additional washings, ELISA plates were developed by adding 50 μl of Substrate-TMB (3,3′,5,5′ Tetramethylbenzidine; Sigma, Cat. No. T0440) in each well for further 30 minutes at room temperature. The chromogenic reaction was stopped by dispensing 100 μl of stop solution (1N Sulphuric acid) into each well and the optical density was read at 450 nm.

Selection of Subcultures of Immortalized Human B Cells that Secrete hCMV Neutralizing Antibodies

The hCMV neutralization assay is sensitive to the presence of even trace amounts of CpG2006. Thus, those cultures that contained IgG antibodies that bound to either the gB or gH regions were gently washed to remove CpG2006 and replaced with medium (IMDM added with 10% FCS and NEAA) without CpG2006 or IL-2. After a further 1-2 weeks of culture, the supernatants were screened using the hCMV neutralization assay described in WO 07/068758.

### Results

Human PBMCs were obtained from a seropositive, asymptomatic blood donor (CMV5) presenting a significant hCMV neutralization titre in serum (50% neutralization at 1:42 dilution), together with a strong reactivity in an ELISA test based on the binding to total hCMV virion proteins. The CMV5 serum was also positive in ELISA tests, showing a weak activity at ¼ dilution when using the gB(AD2) protein (a sample is considered positive for the presence of IgG anti-gB(AD2) at ¼ or higher dilutions), and an activity of 90 AU/ml, when using the total hCMV virion proteins (a sample is considered positive for the presence of IgG anti-hCMV when the result is at least 10 AU/ml).

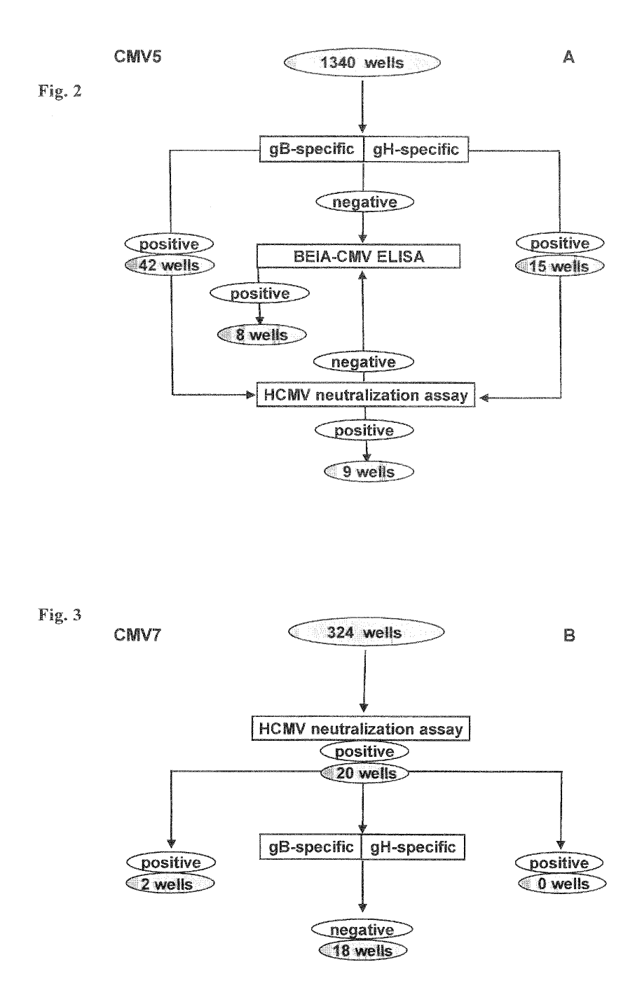

B cells from the CMV5 donor were used for generating an immortalized, polyclonal cell culture highly enriched in B cells that secrete IgG antibodies using the method disclosed in WO 07/068758. Subcultures (3840 samples, each statistically containing 20 cells, for a total of approximately 80000 cells) were prepared from the original polyclonal cell culture and the supernatants selected for the presence of antibodies that bind to regions of the gB or gH envelope glycoproteins of hCMV (FIG. 1) and neutralize hCMV infectivity, as summarized in FIG. 2. A total number of 1340 wells (approximately 35% of the 3840 wells originally plated) contained actively proliferating cells after 3-4 weeks of culture. The supernatants from the 1340 wells were first screened in two ELISAs assays that allow selecting antibodies against specific hCMV envelope glycoproteins. Forty-two wells were positive for gB and 15 wells for gH. The cells from the gB and gH positive wells were then washed to remove CpG2006 and IL-2, cultured for additional 1-2 weeks, and supernatants screened in the hCMV neutralization assay.

Among the 57 supernatants positive for binding to hCMV proteins, 9 were found to reduce the infectivity of hCMV AD169 strain by at least 40%. Those supernatants that were negative in the gB and gH ELISAs, or in the neutralization assay were also screened in an ELISA that detects human IgG that bind to a total hCMV protein extract, and a total of 8 samples were further selected by this screening assay.

Due to the low number of cells initially seeded in each well (20 cells/well), each subculture presenting hCMV-neutralizing activity, should likely produce monoclonal antibodies (i.e. secreted by cells clonally originated by a single, specific immortalized cell), especially given the relatively low frequency of cells in the total B cell population that would be expected to secrete hCMV-neutralizing IgG. Further experimental activities were designed to confirm this assumption.

### Example 2

### Characterization of the 10B7 and 8A11 Antibodies

### Materials and Methods

### Characterization and Expansion of the 10B7 and 8A11 Subculture

The subclass of the antibodies secreted by the 10B7 and 8A11 subcultures was determined using a commercial assay (PeliClass human IgG subclass ELISA combi-kit; cod. RDI-M1551cib, RDI Divison of Fitzgerald Industries Intl.).

The 10B7 and 8A11 cell culture supernatants were tested in immunofluorescence on non-infected HUVEC cells. Briefly, HUVEC cells (7×10⁴/ml) were seeded on gelatine-coated glass-coverslips in 24-well plates in MEM added with 10% FCS and then grown to semi-confluency. Cells were then washed twice with warm PBS and then fixed with a pre-cooled (at −20° C.) mixture of 50% acetone/50% methanol for 1 minute at room temperature (RT) and washed again with PBS. Fixed cells were permeabilized with 0.2% Triton X-100 in PBS for 20 minutes on ice, washed with PBS and incubated for 15 minutes at RT with a blocking solution (PBS added with 2% FCS). Alternatively, fixed cells were not permeabilized to determine the capability of antibodies to recognize cell surface components. In this case, fixed cells were washed with PBS and incubated for 15 minutes at RT with a blocking solution (PBS added with 2% FCS). Then, cells were incubated with the 10B7 or 8A11 cell culture supernatant (80 μl), for 2 hours at 37° C. Cells were then washed with warm PBS (3 times) and incubated with 80 μl of FITC-conjugated rabbit anti-human IgG F(ab′)2 (Jackson ImmunoResearch), to track the human IgG staining as green colour. The secondary antibodies were diluted 1:50 in PBS added with 0.05% Tween80 and left on the cells in the dark for 1 hour at 37° C. Then, cells were washed with warm PBS (3 times) and counter-stained with propidium iodide (Sigma) at a concentration of 0.25 μg/ml in PBS. The coverslips were mounted on microscope slides using one drop of Mounting Medium (Vector Laboratories). Images were recorded with an Olympus Fluoview-IX70 inverted confocal laser scanning microscope.

The cells from the original subcultures 10B7 were expanded on irradiated allogenic PBMC in IMDM medium (added with 10% FCS and NEAA), confirming the hCMV neutralizing activity at least twice during this expansion step using the hCMV microneutralization assay as described in WO 07/068758 and in Example 1. Different combinations of human cells and hCMV strains were used (see Table 1).

The 10B7 culture was gradually expanded by seeding the cells contained in 1 well of a 96-well plate (approx. 1×10⁵) in one well of a 48-well plate on irradiated allogenic PBMC in IMDM added with 5% FCS. After 5-7 days, cells were transferred in one well of a 24-well plate in the absence of feeder layer, in IMDM added with 5% FCS. The amount of antibody secreted by the 10B7 subculture was determined at 24, 48, and 72 hours using a commercial quantitative human IgG ELISA kit (Immunotek; cod. 0801182; Zeptometrix Corp.) according to manufacturer's instructions.

### Characterization of the 10B7 and 8A11 Antibody DNA and Protein Sequence

An aliquot of each cell culture, resulting from the expansion of the initial 10B7 and 8A11 subcultures, was used for sequencing of the variable regions of heavy chain (VH) and light chain (VL) of the 10B7 and 8A11 antibody according to the technology established by Fusion Antibodies Ltd. Briefly, pellets of frozen cells (each containing approx. 50,000 cells) were used for extracting total RNA. The corresponding cDNA was produced by reverse transcription with an oligo(dT) primer. PCR reactions were set up to amplify the VH region using a mix of IgG specific primers, and the VL region with a mix of Igk/λ primers. The PCR products of two amplification reactions were cloned using an Eco RI restriction site in a sequencing vector (pCR2.1; Invitrogen) and used for transforming TOP10 E. coli cells.

At least ten colonies randomly selected from the each transformation were picked and analyzed by sequencing. The resulting DNA sequences were aligned and translated into protein sequence generating a consensus DNA and protein sequence for VH 10B7 (SEQ ID NO.: 4 and SEQ ID NO.: 5, respectively), VL 10B7 (SEQ ID NO.: 9 and SEQ ID NO.: 10, respectively), VH 8A11 (SEQ ID NO.: 14 and SEQ ID NO.: 15, respectively), and VL 8A11 (SEQ ID NO.: 19 and SEQ ID NO.: 20, respectively). The VH 10B7, VL 10B7, VH 8A11, and VL 8A11 protein sequences were compared with sequences present in public databases (GenomeQuest, GeneSeq, and EBI databases). The CDRs characterizing VH 10B7 (SEQ ID NO.: 6, 7, and 8), VL 10B7 (SEQ ID NO.: 11, 12, and 13), VH 8A11 (SEQ ID NO.: 16, 17, and 18), and VL 8A11 (SEQ ID NO.: 21, 22, and 23) protein sequences were predicted by the IMGT database (Lefranc M, 2005).

### Results

The subcultures that were obtained by dividing the bulk culture of immortalized B cells from CMV5 in 20 cell/well populations, were tested for the presence of IgG antibodies that bind to regions of the gB and gH envelope glycoproteins of hCMV using ELISA assays based on gB and gH recombinant antigens (FIG. 1). Alternatively, the subcultures were tested in an ELISA based on a commercially available gB-GST fusion protein containing a gB immunodominant region.

Among the subcultures containing growing and IgG-secreting cells, the cell culture supernatants of a few of them contained antibodies that bind to a region of the gB envelope glycoprotein of hCMV gB-GST fusion protein. In particular, the 10B7 and 8A11 subcultures showed the stronger and more reproducible binding to this fragment of gB (FIG. 3). Therefore, the 10B7 and 8A11 subculture were chosen for a more detailed molecular and biological characterization.

After eliminating CpG2006 from the medium of subcultures, the supernatant from the 10B7 and 8A11 subculture were tested for hCMV neutralizing activity against different hCMV strains in two human host cell systems, in duplicate samples. The results showed an important hCMV neutralizing activity of the antibodies present in the supernatant of these subcultures, in particular for 10B7 antibody which can inhibit hCMV infection using different hCMV strains (Table 1).

Moreover, in order to exclude that the neutralizing activity present in the supernatant from the 10B7 or 8A11 subculture is due to the binding to a surface component on the target cells, the supernatant was tested in immunofluorescence with uninfected HUVEC cells. This assay showed that the IgG antibodies in the supernatant from the 10B7 or 8A11 subculture do not bind to the uninfected human cells, confirming that the 10B7 and 8A11 antibodies (both of IgG1 class) specifically inhibit hCMV infection by binding a neutralizing antigenic domain within the gB envelope glycoprotein of hCMV.

Larger cultures obtained using cells from the 10B7 subculture were generated by gradually expanding the culture and reducing some requirements for growth in cell culture (feeder layer, FCS in the cell culture medium). These cell cultures secrete the 10B7 antibody at a concentration of 11.5 μg/ml/10⁶ cells and showed a doubling time of 4 days, even in the absence of feeder layer. The hCMV-neutralizing activity was confirmed using the supernatant of the larger cell cultures after more than 2 months.

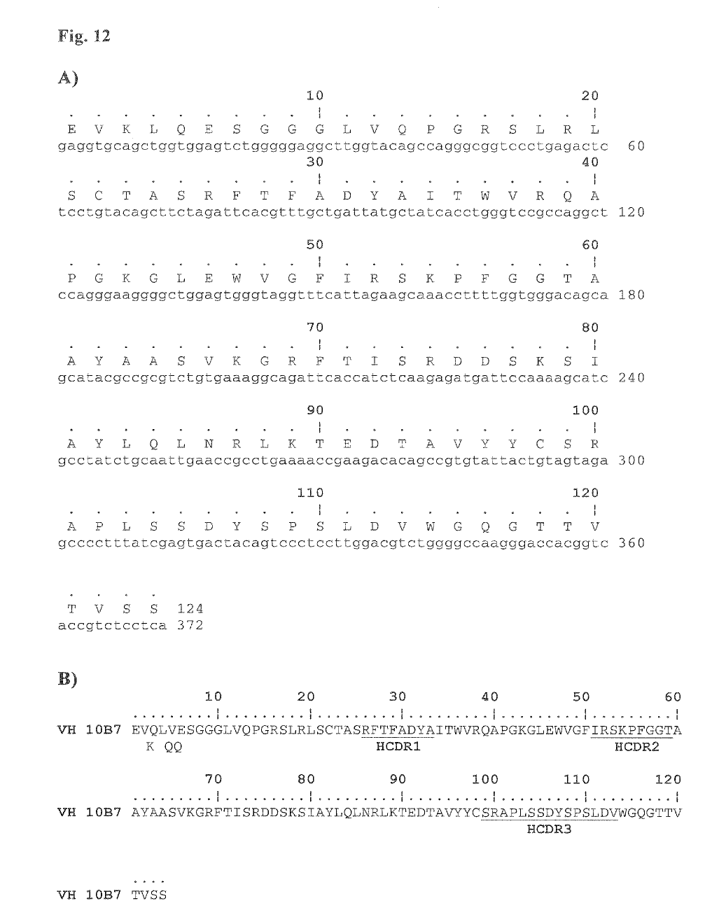

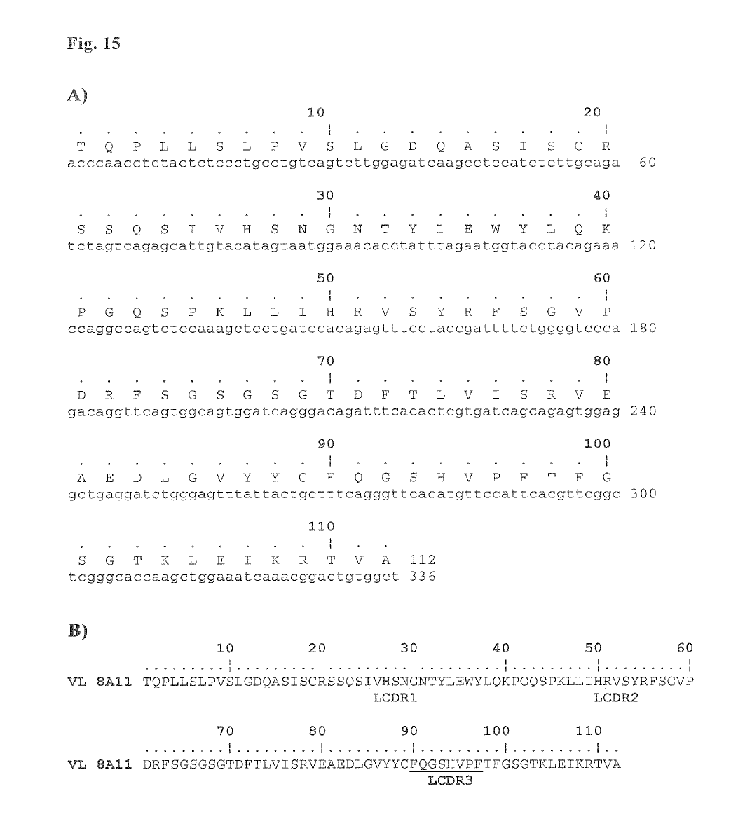

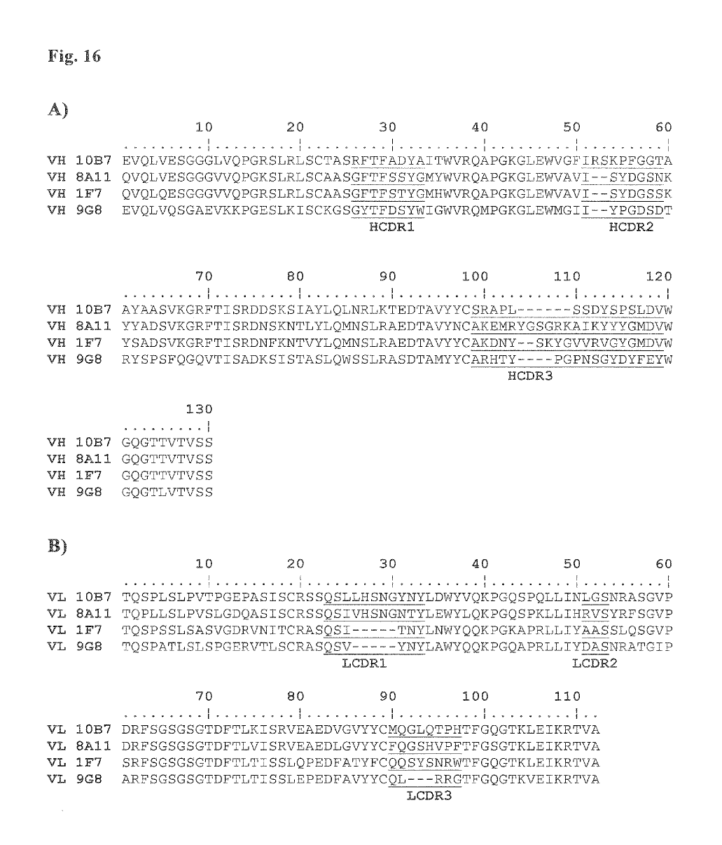

The monoclonality of the hCMV neutralizing antibody secreted in the 10B7- and 8A11-derived cell cultures was also confirmed by sequencing IgG-specific PCR products obtained from this cell culture. Cell pellets were prepared for RNA extraction and reverse transcription using cells originated from the 10B7 or the 8A11 subculture. The resulting cDNA was then used for amplifying VH and VL sequences using specific primers for the variable regions of human IgG heavy and light chain, respectively. The PCR products were then cloned in plasmids that were used for transforming bacterial cells. Bacterial transformants were randomly picked and used for sequencing the cloned PCR products. All the clones showed the same DNA sequence, apart from minor differences possibly due to PCR-induced error, allowing the determination of consensus sequences and CDRs for the variable regions of the heavy chain (FIG. 12) and light chain (FIG. 13) of the 10B7 antibody, of the heavy chain (FIG. 15) and light chain (FIG. 16) of the 8A11 antibody.

The VH and VL sequences of 10B7 and 8A11 antibodies can be aligned with the corresponding sequences for two other antibodies that have been characterized from B cells obtained from the same CMV5 donor in separate screenings. This alignment shows that, even though there are more or less extensive similarities in the CDRs and/or the framework of these sequences, the antibodies are substantially different (FIG. 16).

The sequences encoding the VH and VL regions of the 10B7 or 8A11 antibody can be recloned in expression vectors for the appropriate expression of the 10B7 or 8A11 variable regions as an antibody fragment (Fab or ScFv) or within a fully human, recombinant antibody having a specific isotype and subclass (e.g. IgG1, IgG2, or IgG4). These recombinant antibodies can be tested for confirming the specific hCMV neutralizing activity in the appropriate assays.

TABLE 1

| | hCMV | Human | Inhibition of hCMV infection using |
|---|---|---|---|
| Subculture | Strain | Cell Line | the cell culture supernatant ^{a} |
| 10B7 | AD169 ^{b} | HELF | +++ |
| | VR1814 ^{c} | HUVEC | ++ |
| | AL1 ^{d} | HELF | ++ |
| 8A11 | AD169 ^{b} | HELF | ++ |

^{a} +, ++, +++, and ++++ correspond to 20-40%, 41-60%, 61-80%, and more than 80% of inhibition of the hCMV infection, respectively
^{b} hCMV laboratory strain (from ATCC, code VR-538)
^{c} an endothelial cell-tropic derivative of a clinical isolate recovered from a cervical swab of an hCMV-infected pregnant woman (Revello M et al., 2001)
^{d} a clinical isolate recovered from the bronchoalveolar lavage fluid of a lung transplant recipient

### Example 3

### Production of Cell Cultures Secreting Human Monoclonal Antibodies that Neutralize hCMV

### Materials & Methods

### Production of the Culture of Immortalized Human B Cells

Peripheral blood mononuclear cells (PBMCs) were obtained from a patient from an acute hCMV infection (CMV7) that was selected as presenting hCMV-neutralizing antibodies in the serum. The hCMV-neutralizing antibodies were detected according to an hCMV microneutralization assay based on human Embryo Lung Fibroblasts (HELF cells) and hCMV AD169 strain (an hCMV laboratory strain from ATCC, cod. VR-538). The serum was also tested in an ELISA specific for human IgG binding hCMV virion proteins that is commercially available (BEIA-CMV IgG Quant; Bouty, cod. 21465) and a gB (AD2) hCMV IgG ELISA, also commercially available and described in FIG. 1A (Biotest, cod. 807035, Rothe M et al., 2001) These hCMV-specific assays have been performed as outlined in WO 07/068758 or indicated by the Manufacturer.

The EBV immortalization process to which PBMCs from CMV7 were subsequently exposed has been described in WO 07/068758 and EP07110693. At the end of the process, the immortalized cells were washed with fresh culture medium (RPMI 1640 added with 10% FCS) and put in culture for 3 weeks at a density of 1.5×10⁶ cells/ml in 24 well plates with a feeder layer (irradiated PBMC seeded at 5×10⁵ cells/well). After this expansion phase, the hCMV neutralizing activity was confirmed with the test described above.

Selection of Subcultures of Immortalized Human B Cells that Secrete hCMV Neutralizing Antibodies

Aliquots of the expanded cell culture (each statistically containing 20 cells) were seeded in to 96-well plates on irradiated, allogeneic PBMCs as feeder cells (50,000/well) in 100 μl IMDM (added with 10% FCS and Non Essential Amino Acids (NEAA, diluted 1× from a 100× commercial stock solution; EuroClone), without CpG2006 and IL-2). A total of 4224 cultures were generated and, after two weeks, 50 μl of the same medium were added. After a further 1-2 weeks of culture, the supernatants of cell cultures that presented growing and aggregated cells were tested in parallel using the hCMV neutralization assay based on HELF cells and hCMV strain AD 169 as previously described.

Selection of Subcultures of Immortalized Human B Cells that Secrete IgG Antibodies that Bind to Regions of the hCMV Envelope Glycoproteins gB or gH

The supernatants of cell cultures that presented hCMV neutralizing activity were tested using the ELISAs for detecting binding of human IgG antibodies to regions of the gB or gH hCMV envelope glycoproteins (FIGS. 1A and 1B, respectively; WO 07/068758 and EP07110693).

### Results

Human PBMCs were obtained from an hCMV patient (CMV7) presenting a significant hCMV neutralization titre in serum (50% neutralization at 1:105 dilution), together with a strong reactivity in ELISA based on the binding to the AD2 domain of glycoprotein B (positive at 1:64 dilution), one of the hCMV antigens best characterized as eliciting serum neutralizing antibodies (Qadri I et al., 1992; Kropff P et al., 1993).

B cells from the CMV7 patient were used for generating an immortalized, polyclonal cell culture highly enriched in B cells that secrete IgG antibodies using the method disclosed in WO 07/068758 and EP07110693. Subcultures were then prepared from the original bulk and the supernatants selected for the presence of antibodies neutralizing hCMV infectivity by the microneutralization assay.

As summarized in FIG. 2, out of 4224 wells originally plated, 324 wells contained cells growing at 3 weeks of culture. The supernatants from the 324 wells were first screened in the hCMV neutralization assay, selecting in this manner 20 wells which were found to reduce the infectivity of hCMV AD169 strain by at least 40%. When characterizing the hCMV binding activities in these wells, only two were found positive for gB, none for gH, and 18 wells neither of them.

Due to the low number of cells initially seeded in each well (20 cells/well), each subculture presenting hCMV-neutralizing activity, should likely produce monoclonal antibodies (i.e. secreted by cells clonally originated by a single, specific immortalized cell), especially given the low frequency of cells in the immortalized, polyclonal cell population that is expected to secrete hCMV-neutralizing IgG. Further experimental activities were designed to confirm this assumption.

### Example 4

### Characterization of the 8C10 and 37B7 Monoclonal Antibodies

### Materials and Methods

### Characterization and Expansion of the 37B7 and 8C10 Subcultures

The subclass of the antibodies secreted by the 8C10 and 37B7 subcultures was determined using a commercial assay (PeliClass human IgG subclass ELISA combi-kit; Cell Sciences; Cat. No. M1551).

The 8C10 and 37B7 cell culture supernatants were tested in immunofluorescence on non-infected HUVEC cells. Briefly, HUVEC cells (7×10⁴/ml) were seeded on gelatine-coated glass-coverslips in 24-well plates in MEM added with 10% FCS and then grown to semi-confluence. Cells were then washed twice with warm PBS and then fixed with a pre-cooled (at −20° C.) mixture of 50% acetone/50% methanol for 1 minute at room temperature (RT) and washed again with PBS. Fixed cells were permeabilized with 0.2% Triton X-100 in PBS for 20 minutes on ice, washed with PBS and incubated for 15 minutes at RT with a blocking solution (PBS added with 2% FCS). Alternatively, fixed cells were not permeabilized to determine the capability of antibodies to recognize cell surface components. In this case, fixed cells were washed with PBS and incubated for 15 minutes at RT with a blocking solution (PBS added with 2% FCS). Then, cells were incubated with the 8C10 or 37B7 cell culture supernatant (80 μl), for 2 hours at 37° C. Cells were then washed with warm PBS (3 times) and incubated with 80 μl of FITC-conjugated rabbit anti-human IgG F(ab′)2 (Jackson ImmunoResearch), to track the human IgG staining as green colour. The secondary antibodies were diluted 1:50 in PBS added with 0.05% Tween80 and left on the cells in the dark for 1 hour at 37° C. Then, cells were washed with warm PBS (3 times) and counter-stained with propidium iodide (Sigma) at a concentration of 0.25 μg/ml in PBS. The coverslips were mounted on microscope-slide using one drop of Mounting Medium (Vector Laboratories). Images were recorded with an Olympus Fluoview-IX70 inverted confocal laser scanning microscope.

The cells from the original subculture 8C10 were expanded on irradiated allogenic PBMC in IMDM medium (added with 10% FCS and NEAA), confirming the hCMV neutralizing activity at least twice during this expansion step using the hCMV microneutralization assay as described in WO 07/068758 and in Example 1. Different combinations of human cells and hCMV strains were used (see Table 1 and FIG. 4).

The cell culture was gradually expanded by seeding the cells contained in 1 well of a 96-well plate (≈1×10⁵) in one well of a 48 well plate on irradiated allogenic PBMC in IMDM added with 5% FCS. After 5-7 days, cells were transferred and expanded in one well of a 24-well plate in the absence of feeder layer, in IMDM added with 5% FCS. Then, cells (5×10⁵/ml) were plated in a 6 well plate in the absence of feeder layer in 50% IMDM and 50% Hybridoma-SFM (Gibco, cod. 12045-084) added with 2.5% FCS. Cells were cultured in these conditions for at least one week. Exponentially growing cells were then washed and cultured in T75 Flasks in Hybridoma-SFM at a concentration ranging from 5×10⁵ to 10⁶/ml. Exponentially growing cells were then washed and cultured in T75 Flasks in Hybridoma-SFM at a concentration ranging from 5×10⁵ to 10⁶/ml. Spent medium was collected, the IgG quantified and purified on Protein A columns, dialyzed against PBS buffer and filtered (0.2 μM). The amount of antibody secreted by the 8C10 culture was determined at 24, 48, and 72 hours using a commercial quantitative human IgG ELISA kit (Immunotek; cod. 0801182; Zeptometrix Corp.) according to manufacturer's instructions.

### Characterization of the 8C10 and 37B7 Antibody DNA and Protein Sequence

An aliquot of the each cell culture, resulting from the expansion of the initial 8C10 and 37B7 subcultures, was used for sequencing of the variable regions of heavy chain (VH) and light chain (VL) of the 8C10 and 37B7 antibody according to the technology established by Fusion Antibodies Ltd. Briefly, pellets of frozen cells (each containing at least 50,000 cells) were used for extracting total RNA. The corresponding cDNA was produced by reverse transcription with an oligo(dT) primer. PCR reactions were set up to amplify the VH region using a mix of IgG specific primers, and the VL region with a mix of Igk/λ primers. The PCR products of two amplification reactions were cloned using a Eco RI restriction site in a sequencing vector (pCR2.1; Invitrogen) and used for transforming TOP10 E. coli cells.

At least ten colonies obtained from the two transformations were picked and analyzed by sequencing. The resulting DNA sequences were aligned and translated into protein sequence generating a consensus DNA and protein sequence for VH 8C10 (SEQ ID NO.: 4 and SEQ ID NO.: 5, respectively), VL 8C10 (SEQ ID NO.: 9 and SEQ ID NO.: 10, respectively), VH 37B7 (SEQ ID NO.: 14 and SEQ ID NO.: 15, respectively), and VL 37B7 (SEQ ID NO.: 19 and SEQ ID NO.: 20, respectively). The VH 8C10, VL 8C10, VH 37B7, and VL 37B7 protein sequences were compared and aligned with sequences present in databases in the public domain (using GenomeQuest, GeneSeq, and EBI databases). The CDRs characterizing VH 8C10 (SEQ ID NO.: 6, 7, and 8), VL 8C10 (SEQ ID NO.: 11, 12, and 13), VH 37B7 (SEQ ID NO.: 16, 17, and 18), and VL 37B7 (SEQ ID NO.: 21, 22, and 23) protein sequences were predicted by the IMGT database (Giudicelli V et al., 2006).

### Results

The subcultures that were obtained by dividing the bulk culture of immortalized B cells from CMV7 in 20 cell/well populations, were tested for the presence of neutralizing antibodies in the supernatants by an hCMV neutralization assay and then further tested by additional neutralization assays and selected ELISA assays based on gB and gH recombinant antigens (FIG. 1).

Amongst the subcultures that contain growing and IgG-secreting cells, the cell culture supernatants of a few of them contained antibodies that both neutralize hCMV infection in vitro bind to a region of the gB envelope glycoprotein of hCMV called AD-2. In particular, the 8C10 subculture showed the stronger and more reproducible hCMV neutralization activity and reproducible binding to this fragment of gB. Therefore, 8C10 subculture was chosen for a more detailed molecular and biological characterization, together with another subculture called 37B7 that showed as well a strong hCMV-neutraliazing activity but without a specific binding detected by ELISA (FIG. 3 and Table 1).

Then, in order to exclude that the hCMV-neutralizing activity present in the supernatant from the 8C10 and 37B7 subcultures is due to the binding to a cell surface component, the supernatant was tested in immunofluorescence with uninfected HUVEC cells, This assay showed that the IgG antibodies in the supernatant from 8C10 subculture do not bind to the uninfected human cells, confirming that the 8C10 antibody (an IgG2) specifically inhibits hCMV infection by binding the AD-2 neutralizing antigen within the gB protein. Using this approach it was demonstrated that the IgG antibodies in the supernatant from 37B7 subculture bind to the uninfected human cells. Thus, the 37B7 antibody (an IgG1 antibody) most probably recognizes an undefined antigen on human cell surface that mediates hCMV entry, and that is common to infected and uninfected HELF cells. Different cell surface receptors have been described as being involved in the different phases of hCMV infection, including integrins toll-like receptors and EGF receptors (Wang X et al., 2005; Compton T, 2004). Therefore, even though this neutralizing activity may not be exploited for inhibiting hCMV in vivo, the 37B7 antibody represents a valuable tool for studying hCMV infection and the mechanisms by which antibodies (or any other molecule) can exert an hCMV-neutralizing activity.

The 8C10 and 37B7 supernatant were also tested in two neutralization assays for Herpes Simplex Virus (HSV) -1 and -2, based on HSV-1/-2 mutants expressing LacZ (Laquerre S et al., 1998; Peng T et al., 1998). The supernatants of 37B7 and 8C10 subcultures showed no neutralizing activity in either the HSV-1 or the HSV-2 neutralization assay, confirming the hCMV-specific neutralizing properties of these antibodies.

The cells in the original 8C10 subculture were used for scaling-up IgG production, generating progressively larger cultures from which IgG can be purified and tested in hCMV neutralization assays. Larger cultures obtained using cells from the 8C10 subculture were generated by gradually expanding the culture and eliminating some requirements for growth in cell culture (feeder layer, Fetal Calf Serum in the cell culture medium). Using this approach, it was demonstrated that larger cell cultures generated from the original 8C10 subculture secrete an IgG1 antibody at a concentration of 25 μg/ml/10⁶ cells/day. These larger cultures showed a doubling time of 4 days, even in the absence of feeder layer, and the hCMV neutralizing activity was maintained in culture for more than 2 months.

The hCMV neutralization assays were repeated with the 8C10 antibody, in the form of human IgG2 purified from large cell culture by affinity chromatography. The cell culture-purified 8C10 antibody was tested in dose-response experiments to assess the level of in vitro inhibition of hCMV infectivity, using two assays with different combinations of hCMV strains and human cell lines. The results show that the hCMV-neutralizing activity of cell culture-purified 8C10 antibody is neither cell-type nor hCMV-strain specific (FIG. 4).

The monoclonality of the hCMV neutralizing antibody secreted in the 8C10- and 37B7-derived subcultures was also confirmed by sequencing IgG-specific PCR products. Cell pellets were prepared for RNA extraction and reverse transcription using cells originated from 8C10 and 37B7 subcultures. The resulting cDNA was then used for amplifying VH and VL sequences using specific primers for the variable regions of human IgG heavy and light chain, respectively. The PCR products were then cloned in plasmids that were used for transforming bacterial cells. Bacterial transformants were randomly picked and used for sequencing the cloned PCR products. All the clones showed the same DNA sequence, apart from minor differences possibly due to PCR-induced errors, allowing the determination of consensus sequences and CDRs for the variable regions of the heavy chain (FIG. 5) and light chain (FIG. 6) of the 8C10 antibody, and of the heavy chain (FIG. 7) and light chain (FIG. 8) of the 37B7 antibody.

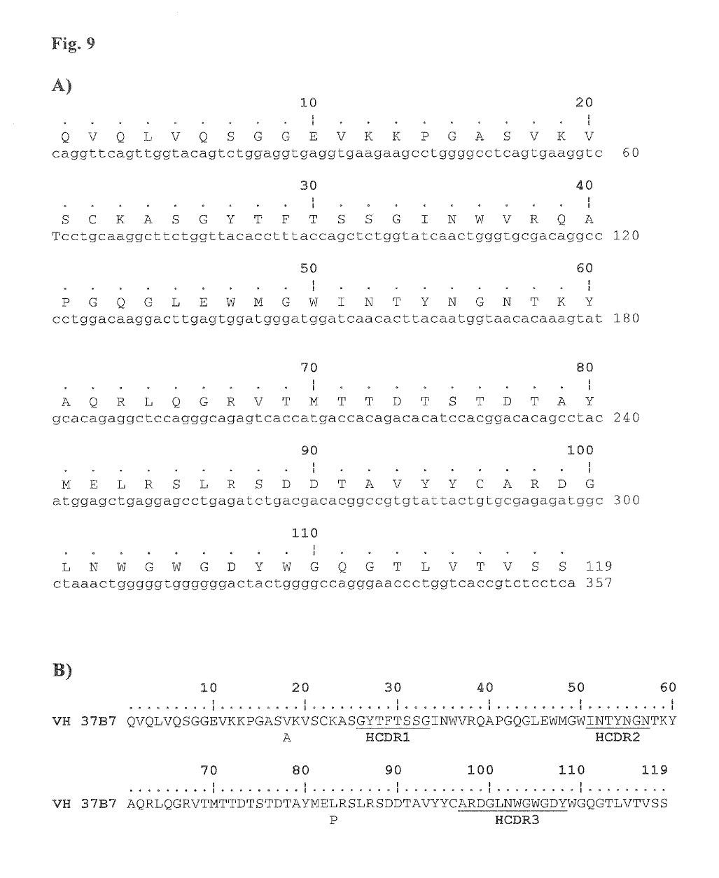

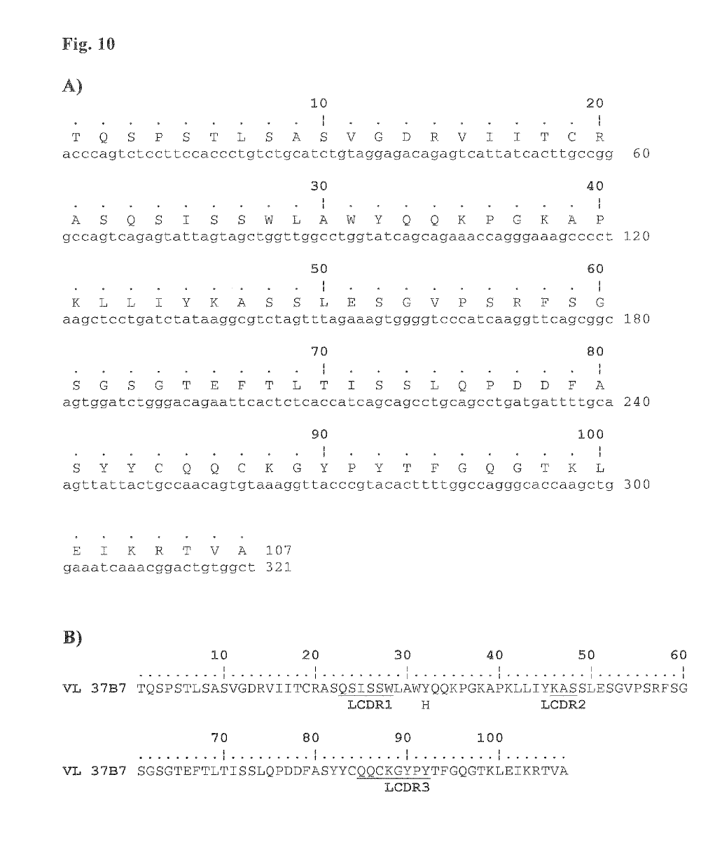

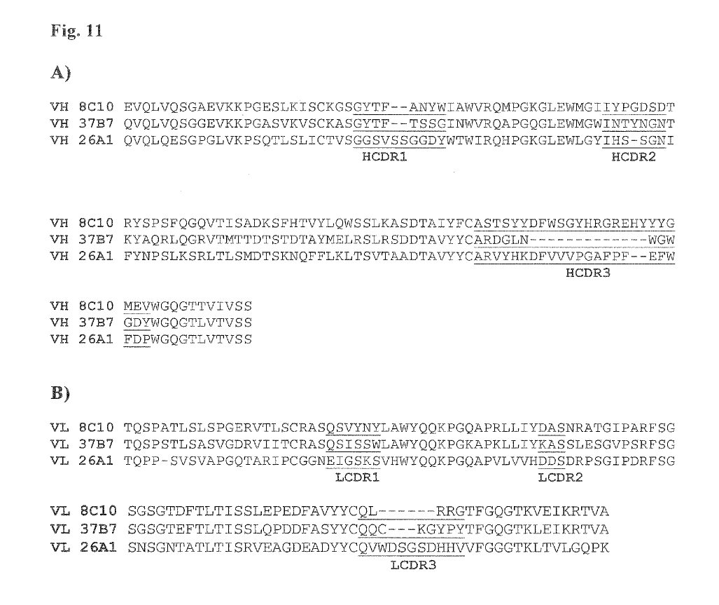

The VH and VL sequences of 37B7 and 8C10 antibodies can be aligned with the corresponding sequences for 26A1, another antibody that have been characterized from B cells obtained from the same CMV7 patient in separate screenings. This alignment shows that, even though there are more or less extensive similarities in the CDRs and/or the framework of these sequences, the antibodies are substantially different (FIG. 9).

The sequences encoding the VH and VL regions of 8C10 and 37B7 antibodies can be recloned in expression vectors for the appropriate expression of 8C10 and 37B7 variable regions as antibody fragments (Fab or ScFv) or within fully human, recombinant antibodies having a specific isotype and subclass (e.g. IgG1, IgG2, or IgG4). These recombinant antibodies can than be tested for confirming the specific binding and hCMV neutralizing activity in the appropriate assays.

TABLE 1

| AntiCMV | hCMV | Human | Inhibition of hCMV infection using |
|---|---|---|---|
| Clone | Strain | Cell Line | the cell culture supernatant ^{a} |
| 8C10 | AD169 ^{b} | HELF | ++ |
| 37B7 | AD169 ^{b} | HELF | ++++ |
| | VR1814 ^{c} | HUVEC | ++++ |

^{a} +, ++, +++, and ++++ correspond to 20-40%, 41-60%, 61-80%, and more than 80% of inhibition of the hCMV infection, respectively
^{b} hCMV laboratory strain (from ATCC, code VR-538)
^{c} a pathogenic endothelial cell-tropic hCMV strain derived from a clinical isolate recovered from a cervical swab of an hCMV-infected pregnant woman (Revello M et al., 2001)

### REFERENCES

Alberola J et al., (2000). J Clin Virol. 16: 113-22.
Aldrich T et al., (2003). Biotechnol Prog. 19, 1433-8.
Baldanti F and Gerna G, (2003). J Antimicrob Chemother. 52: 324-30.
Barrios Y et al., (2004). J Mol Recognit. 17: 332-8.
Barry P et al., (2006). ILAR J. 47: 49-64.
Bianchi A and McGrew J (2003). Biotechnol Bioeng. 84, 439-44.
Boeckh M et al., (2001). Biol. Blood Marrow Tranplant. 7: 343-351.
Bohm E et al., (2004). Biotechnol Bioeng. 88, 699-706.
Bonaros N et al., Transplantation. 2004, 77: 890-7.
Bond C et al., (2003). J Mol Biol. 332: 643-55.
Brereton H et al., (2005). Br J Ophthalmol. 89, 1205-9.
Britt W and Mach M, (1996). Intervirology, 39: 401-12.
Butler M (2005). Appl Microbiol Biotechnol. 68, 283-91.
Carton J et al., (2007). Protein Expr Purif. Doi:10.1016/j.pep.2007.05.017
Castle P et al., (2002). J Reprod Immunol. 56, 61-76.
Chapman A et al., (1999). Nat Biotechnol. 17: 780-3.
Chatenoud L, (2005). Methods Mol Med. 109: 297-328.
Cinatl J et al., (2004). FEMS Microbiol Rev. 28: 59-77.
Coaquette A et al., (2004). Clin Infect Dis. 39: 155-61.
De Clercq E, (2003). J Antimicrob Chemother. 51: 1079-83.
Dinnis D and James D, (2005). Biotechnol Bioeng. 91, 180-9.
Dunman P and Nesin M, (2003). Curr Opin Pharmacol. 3: 486-96.
Eggers M et al., (1998). J Med Virol. 56: 351-8.
Fang J et al., (2005). Nat Biotechnol. 23: 584-90.
Forthal D N et al., Transpl Infect Dis. 2001, 3 Suppl 2:31-4.
Furebring C et al., (2002). Mol Immunol. 38, 833-40.
Gandhi M and Khanna R, (2004). Lancet Infect Dis. 4: 725-38.
Gerna G and Lilleri D, (2006). Herpes. 13: 4-11.
Gilbert G, (2002). Med J Aust. 176: 229-36.
Gilbert C and Boivin G, (2005). Antimicrob Agents Chemother, 49: 873-83.
Gilliland L et al., (1996). Tissue Antigens. 47: 1-20.
Gilpin A et al., (2003). Control Clin Trials, 24: 92-8.
Giudicelli V et al., (2006). Nucleic Acids Res. 34: D781-4.
Goodrum F D et al., (2002).PNAS., 99:16255-60.
Gosselin J et al., (2005). J Immunol. 174:1587-93.
Greijer A et al., J Clin Microbiol. 1999, 37:179-88.
Griffiths P, (2004). Herpes. 11 Suppl 2: 95A-104A.
Griffiths P and Walter S. (2005). Curr Opin Infect Dis. 18: 241-5.
Grote A et al. (2005). Nucleic Acids Res. 33, W526-31.
Grunberg J et al., (2003). Biotechniques. 34, 968-72.
Harris R et al. (2004) Drug Development Research. 61, 137-154.
Hebart H and Einsele H, (2004). Hum Immunol. 65: 432-6.
Horenstein A et al., (2003). J Immunol Methods. 275, 99-112.
Humar A et al., (2003). J Infect Dis. 188: 581-4.
Huse K et al., (2002). J Biochem Biophys Methods. 51, 217-31.
Jain M et al., (2007). Trends Biotechnol.25: 307-16.
Jensen L et al., (2004). J Immunol Methods. 284, 45-54.
Kalil A et al., (2005). Ann Intern Med. 143 870-80.
Keller M and Stiehm E, (2000). Clin Microbiol Rev. 13: 602-14.
Kim S et al., (2005). Mol Cells. 20: 17-29.
Kiss C et al., (2006). Nucleic Acids Res. 34: e132.
Knappik A et al., (2000). J Mol Biol. 296: 57-86.
Kocher A et al., J Heart Lung Transpl. 2003, 22:250-7.
Kropff B et al., (1993). J Med Virol, 39, 187-95.
Kruger R et al., J Heart Lung Transpl. 2003, 22:754-63.
Ladner R, (2007). Nature Biotechnol. 25: 875-77.
Laffly E and Sodoyer R, (2005). Hum Antibodies. 14: 33-55.
Lam V et al., (2006). Biotechnol Bioeng. 93: 1029-39.
Landolfo S et al., Pharmacol Ther. 2003, 98: 269-97.
Lefranc M, (2005). Immunome Res, 1, 3.
Levi M et al., (2000). AIDS Res Hum Retroviruses. 16: 59-65.
Lobo E et al., (2004). J Pharm Sci. 93: 2645-68.
Logtenberg T, (2007). Trends Biotechnol. Doi:10.1016/j.tibtech.2007.07.005.
Ma J et al., (2005). Vaccine. 23: 1814-8.
Matsumoto Y et al., (1986). Bioch Biophy Res Commun. 137: 273-280.
Murray A et al., (2002). J Chromatogr Sci. 40, 343-9.
Navarro D et al., (1997). J Med Virol. 52: 451-9.
Nejatollahi F et al., (2002). FEMS Immunol. Med. Microbiol. 34: 237-244.
Nilsson Jet al., (1997). Protein Expr Purif. 11: 1-16.
Nisnevitch M and Firer M (2001), J Biochem Biophys Methods. 49: 467-80.
Norais N et al., (1996). J Virol. 70: 5716-9.
Ohlin M et al., (1993). J Virol. 67: 703-10.
Ohlin M et al., (1996). Mol Immunol. 33: 47-56.
Qadri I et al., (1992). J Gen Virol, 73 (Pt 11), 2913-21.
Reinhardt B et al., J Virol Methods. 2003, 109: 1-9.
Revello M et al., (2001). J Infect Dis, 184: 1078-81.
Revello M and Gerna G, (2004). J Clin Virol. 29: 71-83.
Rojas G et al., (2004). J Immunol Methods. 293: 71-83.
Roque A et al., (2004). Biotechnol Prog, 20, 639-5.
Rothe M et al., (2001). J Med Virol, 65, 719-29.
Schlatter S et al., (2005). Biotechnol Prog, 21, 122-33.
Schleiss M, (2005). Herpes, 12: 66-75.
Schoppel K et al., (1996). Virology. 216: 133-45.
Schrader J and McLean G, (2007). Doi:1′.1016/j.imlet.200707.001
Smith J et al., (1995). J Biol Chem. 270: 30486-30490.
Snydman D, (2006). Rev Med Virol. 16: 289-95.
Soderberg-Naucler C, (2006a). Crit Rev Immunol. 26: 231-64.
Soderberg-Naucler C, (2006b). J Intern Med. 259: 219-46.
Sorensen H and Mortensen K, (2005). Microb Cell Fact. 4, 1.
Striet M et al., (2006). Int Wound J. 3: 171-9.
Tabrizi M and Riskos L, (2007). Drug Disc Today. 12: 540-7.
Thomsen M et al., (2005). Tissue Antigens. 66: 73-82.
Urban M et al., (1992). J Virol, 66: 1303-11.
Venturi M et al., (2002). J Mol Biol. 315:1-8.
Verdoliva A et al., (2002). J Immunol Methods. 271, 77-8.
Watkins N et al., (2004). Tissue Antigens. 63: 345-54.
Wiegand T and Young L, (2006). Int Ophthalmol Clin. 46: 91-110.
Wijkhuisen A et al., (2003). Eur J Pharmacol. 468: 175-82.
Xu J and Davis M (2000). Immunity. 13: 37-45.
Yoon S et al., (2004). Biotechnol Prog. 20, 1683-8.

1. A protein comprising a sequence having at least 90% identity with SEQ ID NO.: 8.

2. A protein according to claim 1, wherein said protein further comprises SEQ ID NO.: 6 and/or SEQ ID NO.: 7.

3. A protein according to claim 2, wherein said protein comprises a sequence having at least 90% identity with SEQ ID NO.: 5.

4. A protein according to claim 1, wherein said protein further comprises one or more sequences selected from the group consisting of SEQ ID NO.: 11, SEQ ID NO.: 12 and SEQ ID NO.: 13.

5. A protein according to claim 4, wherein said protein comprises a sequence having at least 90% identity with SEQ ID NO.: 10.

6. A protein of claim 1, wherein said protein is an antibody, an antibody fragment, a bioactive peptide, or a fusion protein.

7. A protein of claim 6, wherein said antibody is a human recombinant antibody.

8. A protein of claim 6, wherein said antibody fragment is a variable heavy/light chain heterodimer, or a single-chain fragment variable.

9. The human IgG1 antibody secreted by the 8C10 subculture.

10. A protein of claim 1, wherein said protein binds and neutralizes human Cytomegalovirus (hCMV).

11. A nucleic acid encoding a protein of claim 1.

12. A nucleic acid of claim 11, wherein said nucleic acid comprises a sequence having at least 90% identity with SEQ ID NO.: 4.

13. A nucleic acid of claim 12, further comprising a sequence having at least 90% identity with SEQ ID NO.: 9.

14. A vector comprising a nucleic acid of claim 11.

15. A prokaryotic or an eukaryotic host cell comprising a nucleic acid of claim 11.

16. A host cell of claim 15 wherein said cells secrete a protein of claim 1.

17. (canceled)

18. (canceled)

19. A therapeutic, prophylactic, or diagnostic composition for hCMV infection or for an hCMV-related disease, comprising a protein of claim 1, or a nucleic acid of claim 11.

20. The composition of claim 9 wherein the composition is for ocular or topical administration.

21. A method for the treatment, the prophylaxis, or the diagnosis of hCMV infection, or of a hCMV-related disease, comprising the administration of a protein of 1 or a nucleic acid of claim 11.

22. A method of producing a protein of claim 1 using a nucleic acid of claim 11.

23. A method of producing a protein of claim using a host cell of claim 15.

24. A method of detecting, treating, inhibiting, preventing, and/or ameliorating hCMV infection or a hCMV-related disease using a protein of claim 1.
